SEQUENCE LISTING (12 sequences)

SEQ ID NO: 1 - DNA, 37 bp, Artificial Sequence
  Description of Artificial Sequence Synthetic primer
aggaagagagggtgtaatgtatttatttttgggtttt

SEQ ID NO: 2 - DNA, 51 bp, Artificial Sequence
  Description of Artificial Sequence Synthetic primer
cagtaatacgactcactatagggagaaggctaacaaaccccactccctcat

SEQ ID NO: 3 - DNA, 33 bp, Artificial Sequence
  Description of Artificial Sequence Synthetic primer
aggaagagagttaatgagggagtggggtttgtt

SEQ ID NO: 4 - DNA, 52 bp, Artificial Sequence
  Description of Artificial Sequence Synthetic primer
cagtaatacgactcactatagggagaaggctaacaaactcaccaaaaaaaat

SEQ ID NO: 5 - DNA, 34 bp, Artificial Sequence
  Description of Artificial Sequence Synthetic primer
aggaagagaggttattttttttggtgagtttgtt

SEQ ID NO: 6 - DNA, 52 bp, Artificial Sequence
  Description of Artificial Sequence Synthetic primer
cagtaatacgactcactatagggagaaggctaaaactttcctacccaaaaac

SEQ ID NO: 7 - DNA, 34 bp, Artificial Sequence
  Description of Artificial Sequence Synthetic primer
aggaagagagggtttttgggtaggaaagtttttt

SEQ ID NO: 8 - DNA, 52 bp, Artificial Sequence
  Description of Artificial Sequence Synthetic primer
cagtaatacgactcactatagggagaaggctcctcctttatatcccccaacc

SEQ ID NO: 9 - DNA, 35 bp, Artificial Sequence
  Description of Artificial Sequence Synthetic primer
aggaagagagttgtagtaaagggaaaaggaattga

SEQ ID NO: 10 - DNA, 52 bp, Artificial Sequence
  Description of Artificial Sequence Synthetic primer
cagtaatacgactcactatagggagaaggctccttttcaacaaatattctca

SEQ ID NO: 11 - DNA, 37 bp, Artificial Sequence
  Description of Artificial Sequence Synthetic primer
aggaagagagagtagtttttttgagaatatttgttga

SEQ ID NO: 12 - DNA, 52 bp, Artificial Sequence
  Description of Artificial Sequence Synthetic primer
cagtaatacgactcactatagggagaaggctataccttttaaaaaaaatctt

(19) United States
(12) Patent Application Publication
(10) Pub. No.: US 2011/0190383 A1
(43) Pub. Date: Aug. 4, 2011

(54) Diagnostic, Prognostic and Therapeutic Uses of MIRs in Adaptive Pathways and/or Disease Pathways

(75) Inventors: Clay B. Marsh, Columbus, OH (US); Melissa G. Piper, Dublin, OH (US); Duaa A. Dakhlallah, Columbus, OH (US)

(73) Assignee: THE OHIO STATE UNIVERSITY, Columbus, OH (US)

(21) Appl. No.: 13/119,559

(22) PCT Filed: Sep. 18, 2009
(86) PCT No.: PCT/US09/57432

§ 371 Date: Apr. 20, 2011

Related U.S. Application Data
(60) Provisional application No. 61/098,071, filed on Sep. 18, 2008.
(60) Provisional application No. 61/161,196, filed on Mar. 18, 2009.

(51) Int. Cl. A61K 31/7105; C12Q 1/68; A61P 9/00; A61P 17/00

(52) U.S. Cl. 514/44 A; 435/6.13

(57) ABSTRACT

Described herein are methods and compositions for the diagnosis, prognosis and treatment of various adaptive and/or of disease pathways by examining samples containing one or more miRs therein, and by formulating therapeutic agents therefrom.

### PRIORITY CLAIM AND STATEMENT REGARDING FEDERALLY SPONSORED RESEARCH

This application claims priority to U.S. Provisional Patent Applications Ser. No. 61/098,071 filed Sep. 18, 2008 and Ser. No. 61/161,196 filed Mar. 18, 2009, which are fully incorporated herein by reference. This invention was not made with any government and the government has no rights in this invention.

### BACKGROUND OF THE INVENTION

Various conditions and/or diseases are characterized by injury (and, sometimes, subsequent tissue repair) that transiently or permanently results in changes in adaptive pathways and/or disease pathways. Non-limiting examples of adaptive pathways include one or more of: wound healing, post-surgical recovery, and trauma. Non-limiting examples of disease pathways include one or more of: organ fibrosis such as, but not limited to, cirrhosis, renal fibrosis and injury: solid organ cancer; bone marrow disorders; cardiac fibrosis/failure. A particular pathway is lung fibrosis, including idiopathic pulmonary fibrosis (IPF) associated disease or an interstitial lung disease (ILD).

Idiopathic pulmonary fibrosis (IPF) is an untreatable lung disease caused by repeated episodes of lung injury causing scarring of the lung and chronic inflammation that lead to irreversible thickening of air sacs wall in the lungs. There is no known cure and the progressive nature of this disease ultimately results in a dismal 5 yr mortality rate of 30-50%.

Several investigators have reported increase expression of several genes including CTGF, TNF-α, TGF-β, PDGF, IL-6, IL-10, IL-1β, GM-CSF, Collagen (Col) 1 and 3, and MMPs. However, no mutations are present in the genes to account for their increase in expression, thus the mechanism for their increase is still unknown.

MicroRNAs (miRNAs or miRs) are small single-stranded non-coding RNAs expressed in animals and plants. They regulate cellular function, cell survival, cell activation and cell differentiation during development. MicroRNAs regulate gene expression by hybridization to complementary sequences of target mRNAs resulting in either their inhibition of translation or degradation. MicroRNAs regulate gene expression by targeting messenger RNAs (mRNA) in a sequence specific manner, inducing translational repression or mRNA degradation, depending on the degree of complementarity between miRNAs and their targets (Bartel, D. P. (2004) Cell 116, 281-297; Ambros, V. (2004) Nature 431, 350-355). Many miRs are conserved in sequence between distantly related organisms, suggesting that these molecules participate in essential processes. For example, miRs are involved in the regulation of gene expression during development (Xu, P., et al. (2003) Curr. Biol. 13, 790-795), cell proliferation (Xu, P., et al. (2003) Curr. Biol. 13, 790-795), apoptosis (Cheng, A. M., et al. (2005) Nucl. Acids Res. 33, 1290-1297), glucose metabolism (Poy, M. N., et al. (2004) Nature 432, 226-230), stress resistance (Dresios, J., et al. (2005) Proc. Natl. Acad. Sci. USA 102, 1865-1870) and cancer (Calin, G. A, et al. (2002) Proc. Natl. Acad. Sci. USA 99, 1554-15529; Calin, G. A., et al. (2004) Proc. Natl. Acad. Sci. USA 101, 11755-11760; He, L., et al. (2005) Nature 435, 828-833; and Lu, J., et al. (2005) Nature 435:834-838).

The identification of one or more miRs which are differentially-expressed between normal cells and cells affected by IPF would be helpful. The present invention provides novel methods and compositions for the diagnosis, prognosis and treatment of IIPF.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### SUMMARY OF THE INVENTION

In a first broad aspect, there is provided herein a method of diagnosing or detecting susceptibility of a subject to one or more of a condition characterized by injury and tissue repair that transiently or permanently results in changes in one or more of an adaptive pathways and/or disease pathways.

In certain embodiments, the adaptive pathways include one or more of: wound healing, post-surgical recovery, and trauma.

Also, in certain embodiments, the disease pathways include one or more of: organ fibrosis such as, but not limited to, cirrhosis, renal fibrosis and injury: solid organ cancer; bone marrow disorders; cardiac fibrosis/failure.

In a particular embodiment, the disease pathway comprises lung fibrosis, including idiopathic pulmonary fibrosis (IPF) associated disease or an interstitial lung disease (ILD)

In another broad aspect, there is provided herein a method of diagnosing or detecting susceptibility of a subject to one or more of an idiopathic pulmonary fibrosis (IPF) associated disease or an interstitial lung disease (ILD), comprising;

i) determining the level of at least one miR gene product in the miR-17˜92 cluster in a sample from the subject; and

ii) comparing the level of at least one miR gene product in the sample to a control, wherein an alteration in the level of the at least one miR gene product in the sample from the subject, relative to that of the control, is diagnostic or prognostic of such disease.

In certain embodiments, the miR gene product includes one or more of: miR-17-3p, miR-17-5p, miR-18a, miR-19b and miR-20a.

In certain embodiments, the miR gene product comprises one or more of miR-19a, miR-19b and miR-20a.

In certain embodiments, one or more of the miRs are expressed at low levels in an IPF sample.

In certain embodiments, the control is selected one or more of: a reference standard; the level of the at least one miR gene product from a subject that does not have the disease; and the level of the at least one miR gene product from a sample of the subject that does not exhibit such disease.

In certain embodiments, the subject is a human. In certain embodiments, the alteration is an increase in the level of at least one miR gene product in the sample. In certain embodiments, the alteration is a decrease in the level of at least one miR gene product in the sample.

In another broad aspect, there is provided herein a method of inhibiting progression or proliferation of an idiopathic pulmonary fibrosis associated disorder in a subject, comprising: i) introducing into at least one cell of the subject one or more agents which alter expression and/or activity of at least one miR in the miR-17˜92 cluster within the cell, and ii) maintaining the cells under conditions in which the one or more agents: inhibits expression or activity of the miR; enhances expression or activity of one or more target genes of the miR; or, results in a combination thereof, thereby inhibiting progression or proliferation of the disease or disorder. In certain embodiments, the cell is a human cell.

In another broad aspect, there is provided herein a method of identifying a therapeutic idiopathic pulmonary fibrosis (IPF) agent, comprising:

providing a test agent to a cell and measuring the level of at least one miR in the miR-17˜92 cluster associated with an altered expression levels in the cells,

wherein an alteration in the level of the miR in the cell, relative to a suitable control cell, is indicative of the test agent being a therapeutic agent.

In another broad aspect, there is provided herein a method for regulating levels of one or more proteins in a subject having, or at risk of developing, an idiopathic pulmonary fibrosis (IPF) associated disorder, comprising:

altering the expression of at least one miR gene product in the miR-17˜92 cluster lung cells in the subject.

In certain embodiments, at least one protein comprises: c-myc, CTGF, TSP1, HDAC4.

In certain embodiments, the method includes altering expression of one or more of: miR-19a, miR-19b, and miR-20a.

In certain embodiments, the subject has idiopathic pulmonary fibrosis (IPF).

In certain embodiments, the subject has an interstitial lung disease (ILD).

In another broad aspect, there is provided herein a method for assessing prognosis in a subject with an idiopathic pulmonary fibrosis associated disorder, comprising:

determining a level of at least one miR in the miR-17˜92 cluster which alters expression of one or more of the protein levels of for c-myc, CTGF and HDAC4 as a prognostic indicator of disease progression.

In certain embodiments, at least miR-19b is used be a prognostic indicator of disease state.

In another broad aspect, there is provided herein a method for assessing prognosis in a subject with an idiopathic pulmonary fibrosis associated disorder, comprising:

determining an altered expression of one or more of the protein levels as a prognostic indicator of disease progression,

wherein at least miR-19b and mir20a are down regulated with increasing severity of disease in patients with IPF.

In another broad aspect, there is provided herein a method for altering the expression of a target gene in a subject having, or at risk or developing idiopathic pulmonary fibrosis (IPF), comprising:

inducing expression of one or more miRs in the miR-17˜92 clusters in cells in the subject.

In certain embodiments, the method includes inducing expression by transient transfection in IPF fibroblast cells in the subject sufficient to alter expression of at least one target and/or to change at least one gene networks, to expression those present in normal fibroblast cells.

In certain embodiments, one or more miRs of the miR-17˜92 cluster downregulate expression of one or more genes selected from: CTGF, TGFβ, MMPs, VEGF and thrombospondin-1 (TSP1).

In certain embodiments, the method includes forcing expression of the miR-17˜92 cluster sufficient to downregulate the expression of one or more of the genes and sufficient to downregulate the signaling networks associated therewith.

In another broad aspect, there is provided herein a method for treating idiopathic pulmonary fibrosis (IPF) fibroblasts in lung cells in a subject, comprising introducing one or more miRs in the miR-17˜92 cluster into the cells in an amount sufficient to recover a proliferative and younger phenotype in the cells.

In another broad aspect, there is provided herein a method for enhancing wound healing in lung cells a subject having or at risk of developing idiopathic pulmonary fibrosis (IPF), comprising: transfecting the lung cells with one or more miRs in the miR-17˜92 cluster.

In another broad aspect, there is provided herein a method for treating lung fibroblast cells a subject having or at risk of developing idiopathic pulmonary fibrosis (IPF), comprising:

transfecting the fibroblast cells with one or more miRs in the miR-17˜92 cluster members in an amount sufficient for: i) at least certain of the cells to assume a phenotype similar to non-IPF fibroblast cells; and/or ii) a subsequent increase in expression of one or more proteins selected from: CTGF, TSP-1, MMPs, TGF-beta and VEGF.

In another broad aspect, there is provided herein a method for increasing lung cell development in a subject in need thereof, comprising increasing expression of one or more miRs in the miR-17˜92 cluster in lung cells of the subject.

In another broad aspect, there is provided herein a method for enhancing lung tissue repair and remodeling in response to lung injury in a subject, comprising increasing expression of one or more miRs in the miR17˜92 cluster in lung cells in the subject.

In another broad aspect, there is provided herein a method for treating human idiopathic pulmonary fibrosis (IPF) tissue, comprising increasing expression of one or more miRs in the miR17˜92 cluster in cells in the tissue.

In another broad aspect, there is provided herein a method for altering expansion of marrow precursor cells after lung injury in a subject, comprising increasing expression of one or more miRs in the miR17˜92 cluster in lung cells in the subject.

In certain embodiments, the method of any one of the treatment claims includes the use of miR-19b as the miR selected from the miR-17˜92 cluster.

In certain embodiments, at least one other miR in the miR-17˜92 cluster is used in combination with miR-19b for therapeutic impact.

In another broad aspect, there is provided herein a method for detecting changes in myofibroblast production and/or detecting alterations in epithelial cell-to-mesenchymal cell transition in a subject having, or at risk of developing idiopathic pulmonary fibrosis (IF), comprising: measuring levels of one or more miRs in the miR17˜92 cluster in lung cells in the subject.

In certain embodiments, at least one of miR-19b and miR-20a are down regulated with increasing severity of disease in patients with IPF.

In another broad aspect, there is described herein method of detecting susceptibility of a subject to an idiopathic pulmonary fibrosis (IPF) associated disease, comprising: i) determining the level of at least one miR gene product selected from the miR-17˜92 cluster in a sample from the subject; and ii) comparing the level of at least one miR gene product in the sample to a control, wherein an increase in the level of the at least one miR gene product in the sample from the subject, relative to that of the control, is diagnostic or prognostic of such disorder.

In certain embodiments, the control may be one or more of: a reference standard; the level of the at least one miR gene product from a subject that does not have the disorder; and iii) the level of the at least one miR gene product from a sample of the subject that does not exhibit such disorder. In certain embodiments, the subject is a human. In a particular embodiment, the alteration is a decrease in the level of the miR gene product in the sample.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1—Table showing upregulated miRs in human IPF.

FIG. 2—Table showing downregulated miRs in human IFP.

FIG. 3—Hierarchical cluster analysis of miRs in lung tissue from patients with interstitial lung disease ((ILD) and normal tissue (CTRL).

FIG. 4—Graph showing the validation of expression of miR-19b in ILD tissue.

FIGS. 5A-5C—Graphs showing the validation of miR expression in human idiopathic pulmonary fibrosis (IPF) v. control (CTRL) by quantitative RT-PCR

FIG. 6—Graph showing miR-17˜92 expression in human lung fibroblast.

FIG. 7—Comparison between normal lung fibroblast (left) and IPF lung fibroblast (right) for: Ets-2, TGFβ, Elk3, E2F1, CTGF, Tsp-1 and β-action.

FIG. 8—Graph showing the miR-17˜92 cluster expression in human IPF samples.

FIG. 9—Table showing the microRNAs involved in regulating gene expression involved in IPF.

FIGS. 10A-10B—Hierarchical clustering of gene expression profiles from IPF/ILD, COPD, and control (CTRL) samples. All tissue samples were obtained from the LTRC or CHTN. RNA was isolated and profiled by Affymetrix gene chips.

FIG. 10A—Unsupervised clustering of mRNA profiles from 21 patients with IPF/ILD, 6 patients with COPD, and 5 controls (uninvolved lung tissue from patients undergoing surgery for lung cancer). The unsupervised clustering was applied to the gene expression profiles after a one-way ANOVA test. The program, Bioconductor, was used for this analysis.

FIG. 10B—IPF/ILD profiles clustered with themselves after a 2-way ANOVA test. The FVC group ILD-1 (<50% FVC), ILD-2 (50-80% FVC), or ILD-3 (>80% FVC, least severe breathing impairment) is shown as the last digit of the sample identification. At least some of the IPF/ILD patients falling into groups 1, 2, or 3 clustered together by this analysis.

FIGS. 10D-10E—IPF/ILD patients with distinct forced vital capacity have different patterns of gene expression:

FIGS. 10C-10D—Increased expression of VEGF (FIG. 10F) and CTGF (FIG. 10G) according to disease severity. Real-time PCR reaction was performed for VEGF and CTGF. Shown is the average relative expression normalized to 18 s internal control±S.E.M (Control n=3, >80% FVC n=4, 50-80% FVC n=4, and <50% FVC n=4).

FIG. 10E—Biological pathways implicated in ILD: preliminary comparison of ILD profiles relative to control profiles. The mean expression value for each gene within a sample grouping (IPF/ILD or CTRL) was fit into an analysis of variance model. Confidence intervals were calculated across all results using Tukey's Honest Significant Differences calculation in R/Bioconductor, producing an adjusted p-value. The 10 pathways with the highest significance are shown.

FIGS. 11A-11C—Graphs showing the decreased expression of the miR-17˜92 cluster in lung tissue from FVBM mice treated with bleomycin (Bleo).

FIGS. 11D-11E—Pathological and protein assessment of bleomycin-induced fibrosis in mice for PBS and Bleomycin.

FIGS. 12A-12B—Graphs showing changes in expression of the miR-17˜92 cluster in bleomycin-induced fibrosis in C57BL/6 mice, as compared with PBS samples.

FIG. 13—Graph showing IPF gene expression in bleomycin treated C67BL/6 mice.

FIGS. 14A-14K—graphs showing the effect of over-expression of miR-17˜92 cluster on IPF gene expression for Tsp-1, VEGF, Elk3, HIF1A, TN-C and HIF1B, Ets-2, Ets-1, CTGF, Col13a and Col1a; left-to-right: Untreated normal lung fibroblast; Normal+MiR-17˜92 cluster (0.5 ug); Normal+miR-17˜92 cluster (1.0 ug); Untreated IPF lung fibroblast; IPF+miR-17˜91 cluster (0.5 ug); IPF+miR-17˜92 cluster (1.0 ug).

FIGS. 15A-15B—Re-introduction of the miR-17˜92 cluster in IPF-derived lung fibroblasts decreases expression of VEGF and CTGF. Cells were transfected with either empty vector (pcDNA3.1) or the pcDNA3.1/miR-17˜92 expression vector using Effectene then cultured for 48 h. RNA was isolated and then subjected to qRT-PCR using specific primers to VEGF (FIG. 15A) or (B) CTGF (FIG. 15B) and 18 s was used as an internal control. Fold change compared to untransfected cells was determined. Data shown is the average±SEM (n=3).

FIG. 16—miR-17˜92 transfection induces phenotypic changes in lung fibroblasts derived from patients with IPF. IPF-derived lung fibroblasts were transfected with the miR-17˜92 cluster. Equal cell numbers for untransfected (IPF) and transfected (IPF+17-92 cluster) cells were cultured and photographed daily to visualize phenotypic changes.

FIGS. 17A-17U—Graphs for gene expression in human IPF patient samples based on disease severity; left-to-right: Normal (n=3); group 3>80% (n=4); group 2˜50-80% (n=4); group 1<50%, showing the Relative Expression 2̂(−dCT): FIG. 17A=IL-6; FIG. 17B=Map3k; FIG. 17C=Mmp-7; FIG. 17D=SOCS-3; FIG. 17E=FAS; FIG. 17F=FN-1; FIG. 17G=TSC-2; FIG. 17H=SOX-17; FIG. 17I=THB-1; FIG. 17J=IL-1-R2; FIG. 17K=Ets-2; FIG. 17L=Ets-2; FIG. 17M=Col-1; FIG. 17N=Elk-3; FIG. 17O=Tert; FIG. 17P=Col-3; FIG. 17Q=Col-13a; FIG. 17R=LBTP; FIG. 17S=CTGF; FIG. 17T=VEGF.

FIG. 18—Protein expression in lung tissue from patients with IPF.

FIGS. 19A-19B—MiR19b expression in human lung tissue.

FIGS. 20A-20B—MiR1920a and Let-7 expression in human lung tissue.

FIGS. 21A-21D—Graphs showing the miRNA expression in human lung fibroblast cell lines.

FIG. 22—Protein expression in human normal and IPF lung fibroblast cell lines, where N=Normal, and I=IPF.

FIGS. 23A-23B—Morphology for human lung fibroblasts in Normal and IPF.

FIGS. 24A-24B—IPF-derived fibroblasts transfected with the miR-17˜92 cluster begin to assume a phenotype similar to normal lung fibroblasts.

FIGS. 25A-25B—Overexpression of the miR-17˜92 cluster in normal lung fibroblasts does not alter their phenotype.

FIGS. 26A-26B—Overexpression but not knockdown expression of miR-19b or miR-20a induces phenotypic changes in IPF lung fibroblast cell lines.

FIGS. 27A-27B—Knockdown expression of miR-19b or miR-20a induces normal lung fibroblast cell lines to become phenotypically similar to the IPF lung fibroblast cell lines.

FIGS. 28A-28D—Confirmation of miR-19b and -20a expression in human lung fibroblasts after transfections.

FIGS. 29A-29J—Increased gene expression in both normal and IPF fibroblast cell lines when expression of either miR-19b or miR20a is knockdown.

FIG. 30—Decreased protein expression following transfection of miR-17˜92 in IPF lung fibroblast cell lines was found, where U=Untransfected, M=Mock transfection, V=Empty vector, and C=miR-17˜92 cluster.

FIG. 31—Decrease protein expression following transfection of either miR-19b or 20a in IPF lung fibroblast cell lines, where U=untransfected, Sc=scramble control, +20=miR-20a, 20=miR-20a antagomirs, +19=miR-19b, and 19=miR-19b antagomirs.

FIGS. 32A-32B—The Location of CpG islands in the promoter of miR-17˜92 and primer sequences used for DNA methylation studies.

FIG. 33—Increased DNA methylation of miR-17˜92 promoter in IPF tissue and fibroblast cell lines compared to normal tissue and cells.

### DETAILED DESCRIPTION OF THE PREFERRED EMBODIMENT

The present invention is based, in part, on the identification of specific microRNAs (miRNAs) that are involved in an inflammatory response and/or have altered expression levels. The invention is further based, in part, on association of these miRNAs with particular diagnostic, prognostic and therapeutic features.

In IPF, proteins involved in abnormal wound repair leading to scarring of the lung are increased. There are no known genetic mutations to explain for these changes in protein expression. The inventors herein now show that a decrease in expression of regulatory microRNAs occurs to account for these alterations.

The microRNA cluster miR-17˜92 encodes seven microRNAs (miR-17-Sp, miR-17-3p, miR-18, miR-19a, miR-19b, miR-20a, miR-92). The expression of each individual microRNA contained within the miR-17˜92 cluster from patients with IPF by quantitative RT-PCR as well as a mouse model was examined. Expression of miR-19b decreased in both mice and human pulmonary cells. Also, expression of miR-19b decreased proportionately with severity of disease in humans, thus showing that at least miR-19b is useful as a biomarker for IPF and as a therapeutic target and/or agent for IPF.

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference. The following examples are intended to illustrate certain preferred embodiments of the invention and should not be interpreted to limit the scope of the invention as defined in the claims, unless so specified. In particular, the value of the present invention can thus be seen by reference to the Examples herein.

### Example 1

### Up- and Down-Related miRs

The inventors herein determined which miRNAs regulate the expression of genes that are known to be upregulated in IPF. Several of these genes are regulated by miRNAs that are found in the miRNA cluster, miR-17˜92. This cluster encodes seven microRNAs (miR-17-5p, miR-17-3p, miR-18, miR-19a, miR-19b, miR-20a, miR-92).

Since TSP-1 is an activator of TGF-β and since CTGF and TGF-β are elevated in IPF, the inventors determined whether the expression of the miR-17˜92 is decreased in IPF.

RNA isolated from human lung biopsies from patients with IPF were subjected to microRNA transcriptional profiling. From human IPF lung tissue, a significant decrease in expression of 23 known microRNAs was identified. A greater than 80% decrease in expression of miR-17, miR-19b and miR-20 encoded from the miR-17˜92 cluster was detected.

A greater than a two-fold increase in the expression of 83 microRNAs and five microRNAs was found, had a greater than 100-fold increase.

To directly examine the expression of the miR-17˜92 cluster, quantitative PCR was performed using specific primers to each of the microRNAs within the cluster. A 30-50% decrease in expression of miR-17, miR-19a, and miR-19b was found.

Data using a murine model of fibrosis to examine the miR-17˜92 cluster showed that expression of both miR-19a and miR-19b are decreased. The data also showed that the changes in gene and protein expression in IPF are due to abnormal microRNA regulation. Understanding the microRNAs involved in the development and progression of IPF will enable the design of novel therapies in IPF.

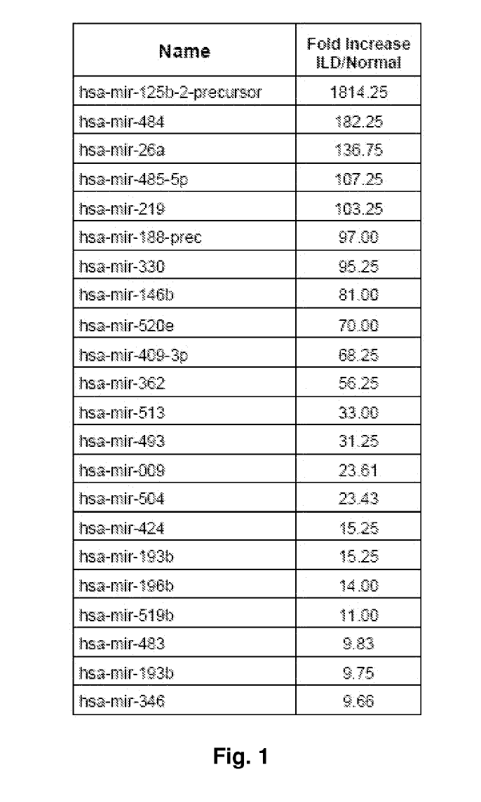

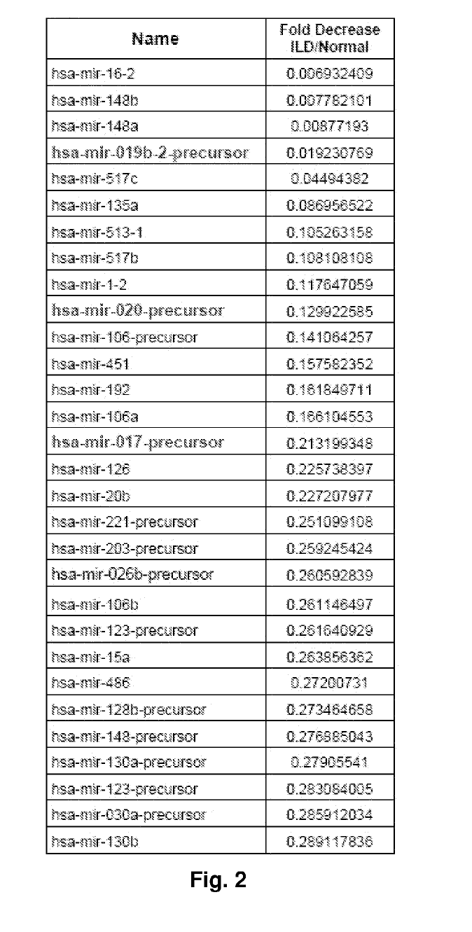

FIG. 1 and FIG. 2 show the total RNA from normal (NL) lung tissue or lung tissue from patients with interstitial lung disease (ILD)/IPF were subjected to miRNA transcriptional profiling. Relative expression of the microRNAs is shown as a ratio of ILD/normal. Highlighted miRNAs correspond to the miR-17˜92 cluster present on the chip. Shown is the average from two different donors per each group.

The expression of each of the miRNAs contained within the miR-17˜92 cluster in a mouse model of pulmonary fibrosis was analyzed. FIG. 1 and FIG. 2 show the decrease expression of miR-19b in a mouse model of pulmonary fibrosis. MiRNA expression was examined from lung tissue from bleomycin-treated mice or vehicle (PBS) control treated mice by quantitative RT-PCR. Relative expression was normalized to 18 s RNA control. Shown is the average±S.E.M, from eight mice per group.

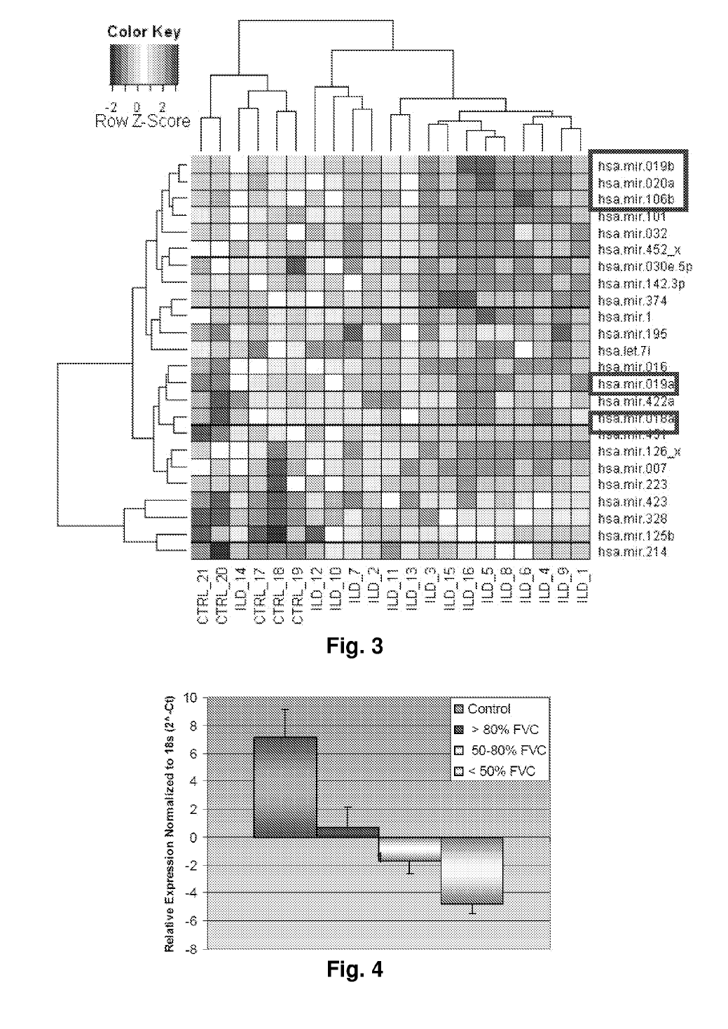

The inventors then determined whether this decrease occurs in humans with IPF. MicroRNA expression profiles from patients with interstitial lung disease (ILD)/IPF and control (CTRL) lung tissue were analyzed. The ILD/IPF lung tissues were divided into three categories according to severity of disease based on forced vital capacity (FVC): group 1<50% (most severe); group 2, 50-80%; and group 3, >80%. The unsupervised hierarchical clustering results for 16 ILD/IPF patient samples and 5 control samples are shown in FIG. 3. The majority of the control and IPF samples had similar expression profiles as indicated with the samples clustering together. Notably, a decrease in expression of miR-019b and miR-020a in the ILD/IPF samples was detected, as compared to the control tissue. FIG. 4 shows the hierarchical cluster analysis of miRNAs in lung tissue from patients with ILD and normal tissue (CTRL). Correction of the raw PCR cycle threshold (CT) scores included geometric mean normalization. There were 24 miRNAs that were differentially expressed among the samples as determined by Student's t-test followed by the Benjamini & Hochberg multiple test correction. Color key: dark green, highest expression; dark red, lowest expression.

Since a similar decrease of miR-19b in both mouse and human pulmonary fibrosis samples was observed, this decrease was further validated. Validation of decrease targets involved using increase RNA for the quantitative RT-PCR. Interestingly, a consistent decrease in miR-19b is apparent with increasing severity of IPF, suggesting a potential marker of disease progression.

FIG. 4 shows the validation of expression of miR-19b in ILD tissue. To confirm the observed decrease in miR-19b expression from profiling data, total mRNA was increased in the real-time PCR reaction by 100-fold. The reaction was repeated using a different Applied Biosystems 7900HT real-time PCR instrument and a 96 well format. Shown is the average relative expression normalized to 18 s internal control±S.E.M (Control n=6, >80% FVC n=8, 50-80% FVC n=6, and <50% FVC n=3). These data show that the loss of miR-19b expression has a potential to predict disease.

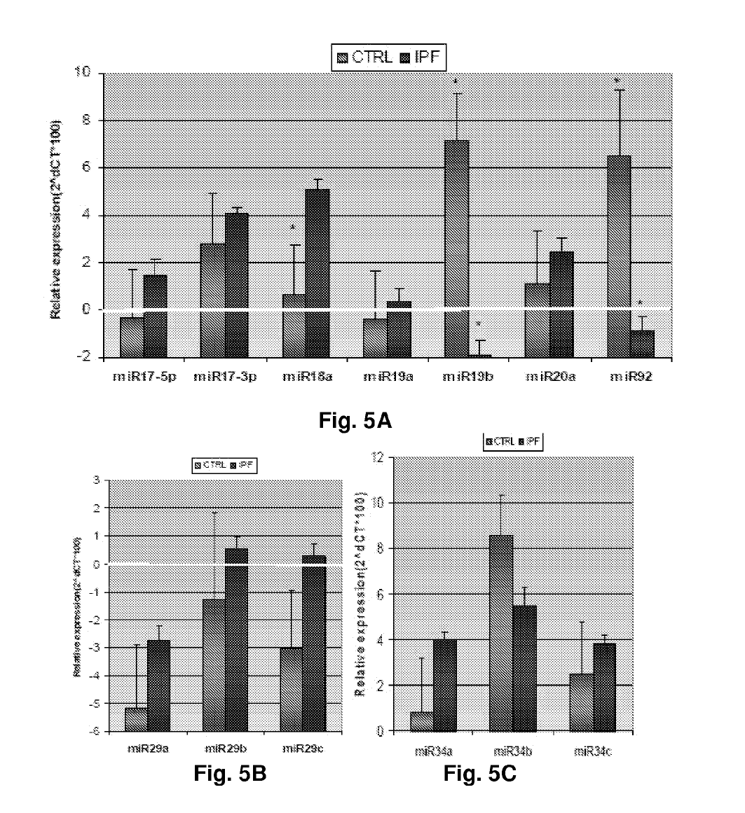

FIG. 5A-FIG. 5C show the validation of microRNA expression in human IPF by quantitative (q)RT-PCR. RNA from control (CTRL) or interstitial lung disease (ILD)/IPF lung tissue was subjected to qRT-PCR using specific primers of each microRNA. Relative expression was determined using 18 s RNA as an internal control. FIG. 5A shows the expression of the miR-17˜92 cluster in IPF. FIG. 5B shows the increase the expression of miR-29 family in IPF samples compared to control samples. FIG. 5C shows the expression of miR-34 family in IPF samples. Data shown are the average±SEM (CTRL n=6, IPF n=17). P<0.05.

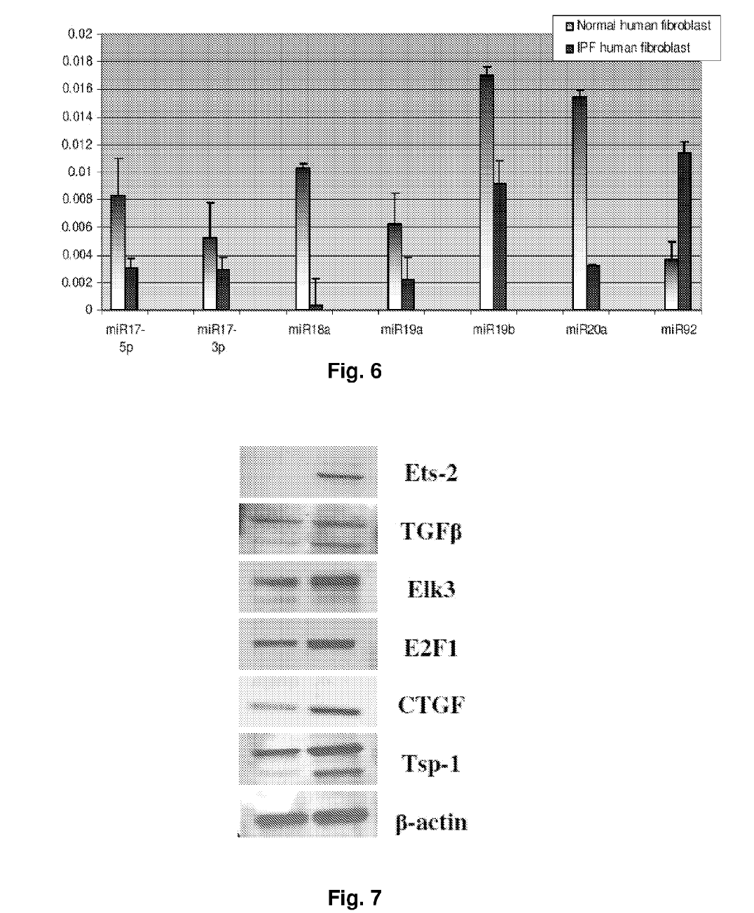

FIG. 6 is a graph showing miR-17˜92 expression in human lung fibroblast.

FIG. 7 shows a comparison between normal lung fibroblast and IPF lung fibroblast for Ets-2, TGFβ, Elk3, E2F1, CTGF, Tsp-1 and β-action.

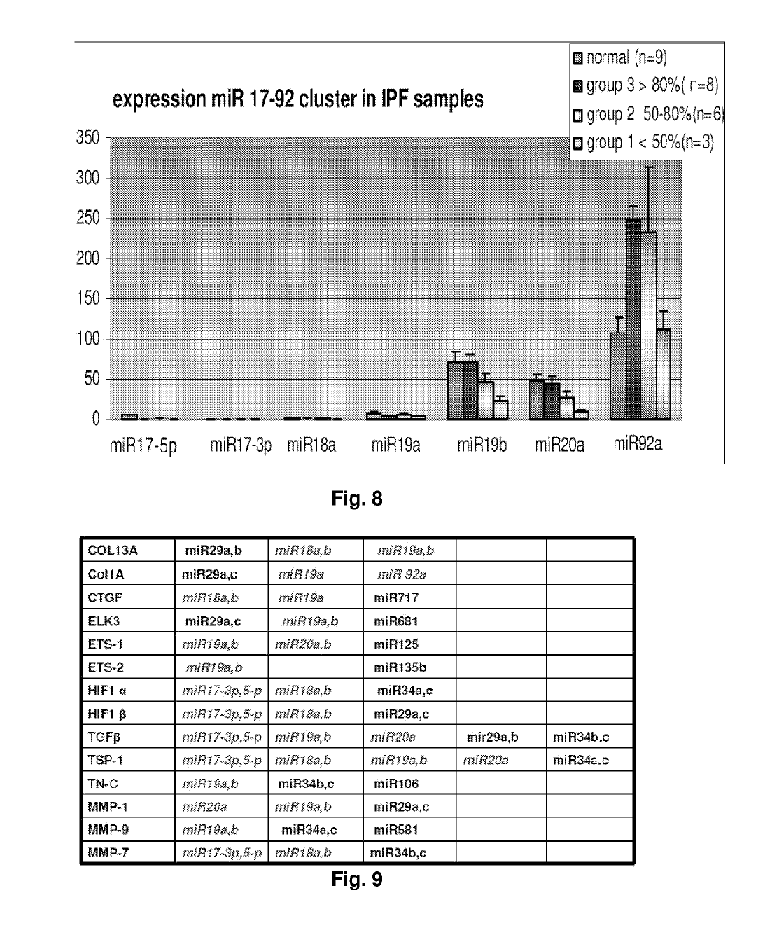

FIG. 8 is a graph showing miR-17˜92 cluster in IPF samples, and in particular, the miR-19b expression and miR20a expression.

Also, expression of miR-34b was decreased but the other miR-34 family members or miR-29 cluster are not, suggesting that these microRNAs do not play a major role IPF.

Further, while protein levels for c-myc and CTGF were increased, HDAC4 was decreased in the mouse model of pulmonary fibrosis. Also, HDAC4 expression can be regulated by miR-17-5p, miR-20a, and miR-19a, all of which are increased in both human and mouse pulmonary fibrosis. Also, that miR-19b is useful as a prognostic indicator of an IPF disease state, as well as at a target for therapy for IPF.

### Example 2

### mRNA Profiling of IPF Patients

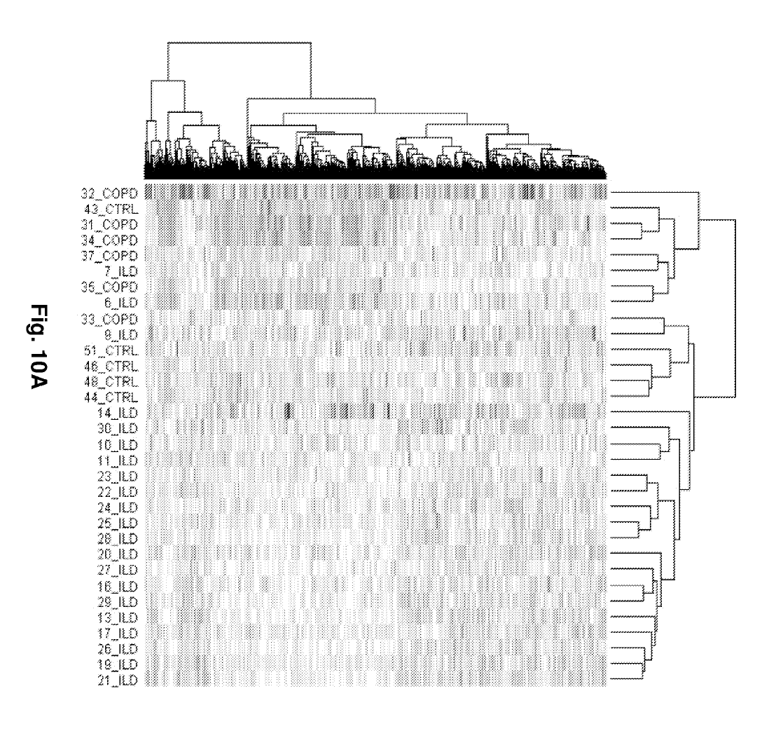

To determine novel targets in pulmonary fibrosis, lung tissue was obtained from the Lung Tissue Research Consortium and these patients were stratified by a number of different quantitative metrics, including lung function testing. Distinct mRNA expression profiles distinguishing patients with IPF/ILD from controls (normals and COPD samples) were found. FIG. 9 shows the microRNAs which regulate gene expression involved in IPF.

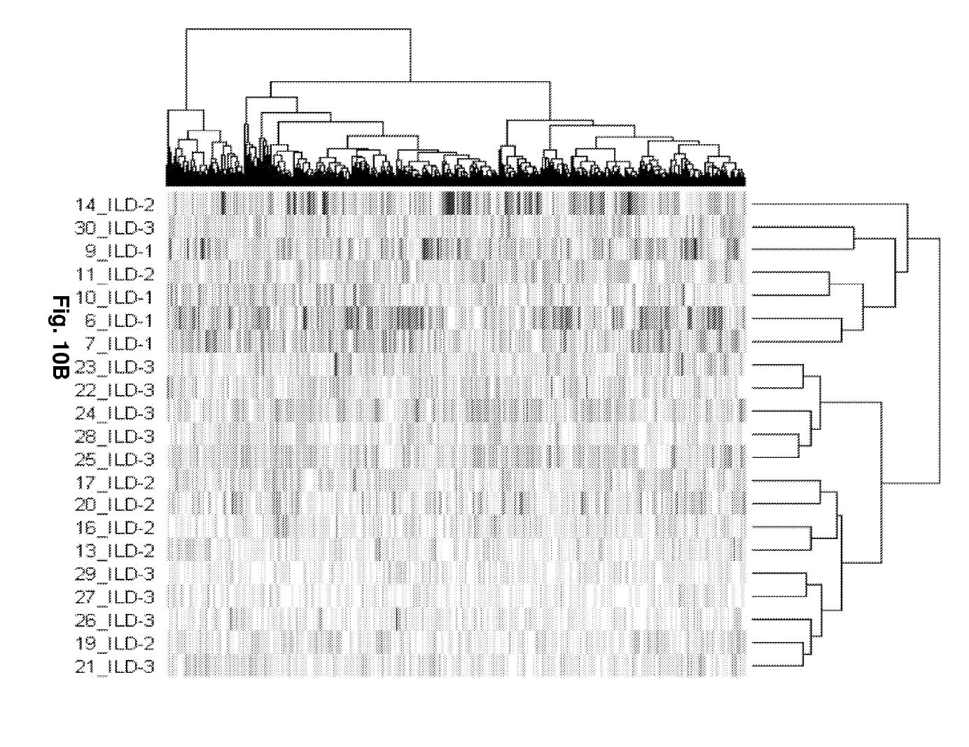

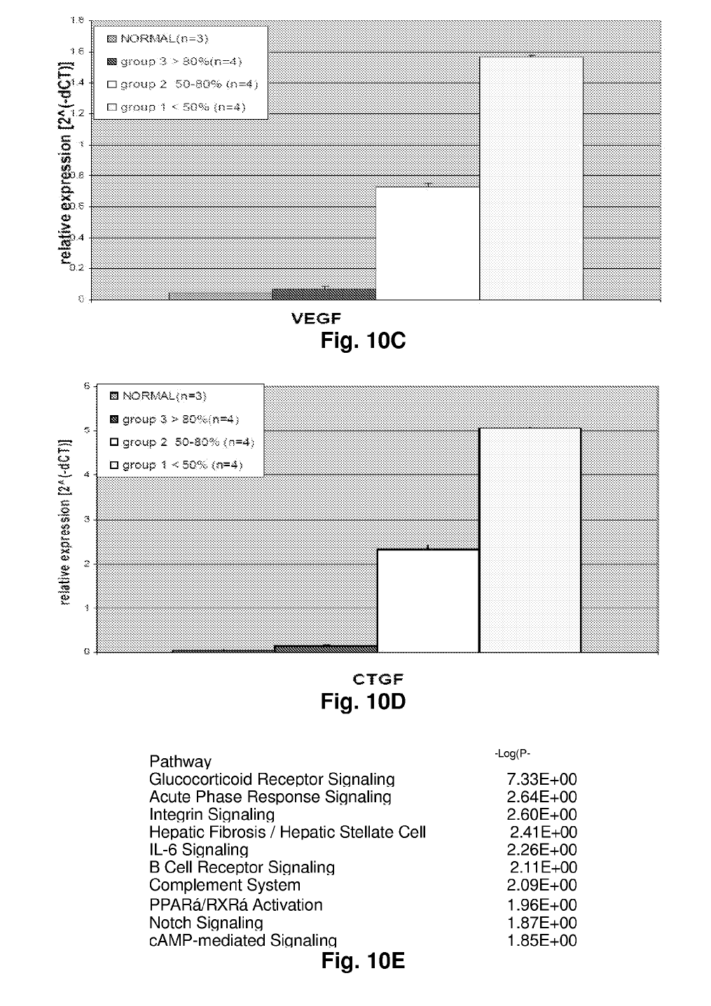

As shown in FIG. 10A, the unsupervised cluster analysis resulted in 18 of the 21 profiles from IPF/ILD patients grouped together (on the right side of the figure), while 4 of the 7 COPD profiles were grouped together on the left of the figure. Quantitative phenotyping data were used to stratify the data, including stratification by the forced vital capacity (FVC pre-bronchodilator, % predicted). Using this phenotype to stratify the profiled lung tissue, we saw natural mRNA clustering of IPF/NSIP patients compared to normals and COPD lung tissue mRNA profiles (FIG. 10B). There was a clear tendency for at least some of the IPF/ILD patients with similar FVC scores to group together based on gene expression profiles. Quality assurance checks included examination of RNA integrity, cDNA yield after amplification, visual inspection of the Affymetrix raw data files (for a high background or other hybridization artifact), and study of the final profiles for outliers.

Several genes elevated in patients with IPF include CTGF and VEGF and the expression of these genes in the patient samples was examined. As shown in FIG. 10C-FIG. 10D, both VEGF and CTGF increased in expression with worse disease. The highest expression level was observed in the most severe cases (<50% FVC).

### Example 3

### Bleomycin Treatment

While an increase in the expression of several of the miRNAs (miR-19a and miR-20a) was observed, a significant decrease in the expression of miR-19b in mice treated with bleomycin compared to control mice was found. Also, an increase in CTGF protein expression in the lung from bleomycin-treated mice was found.

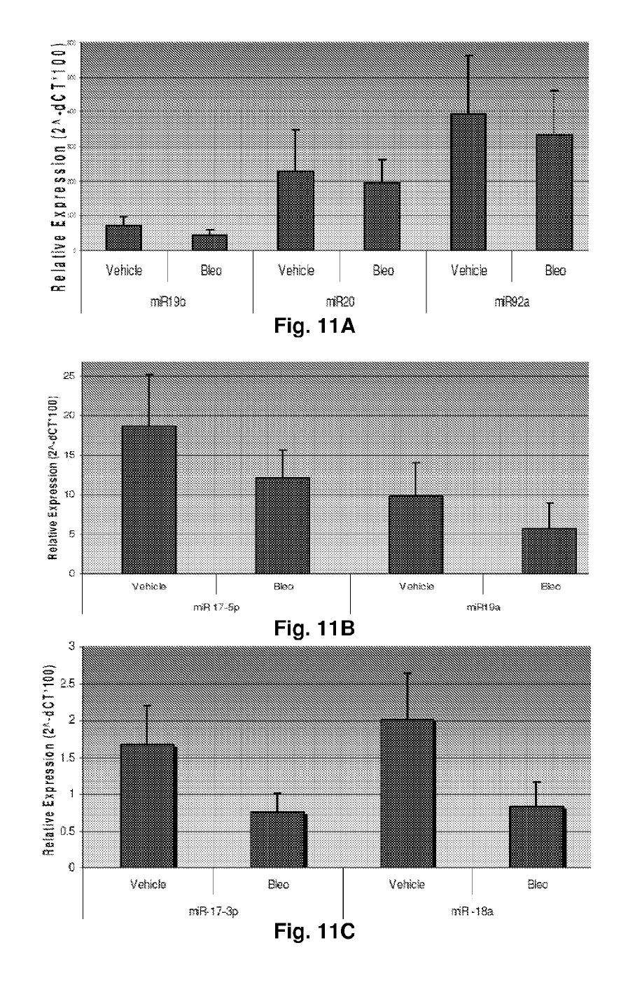

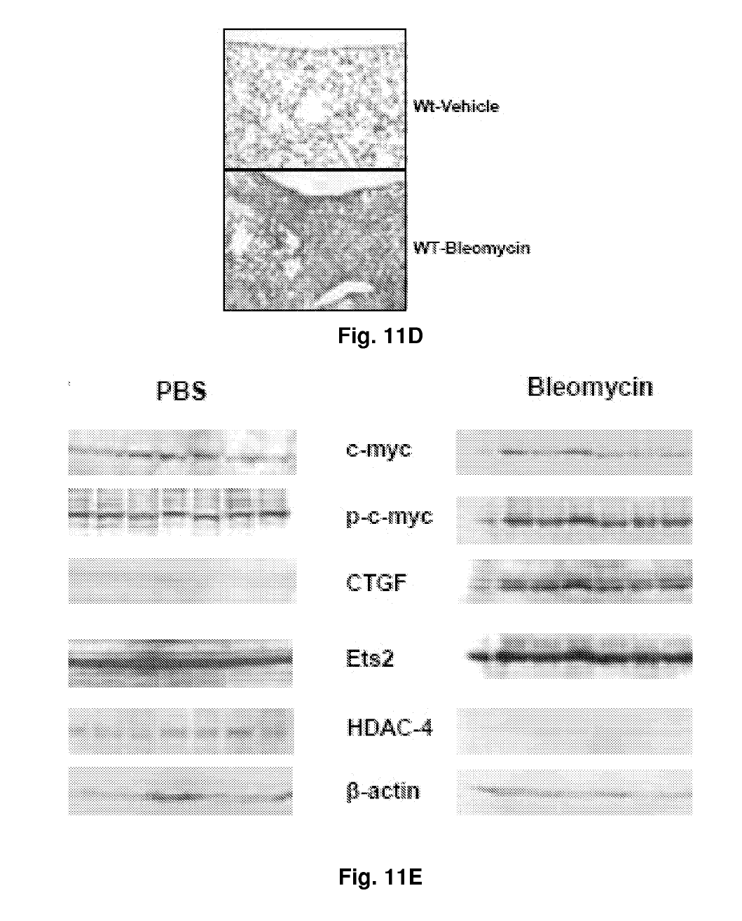

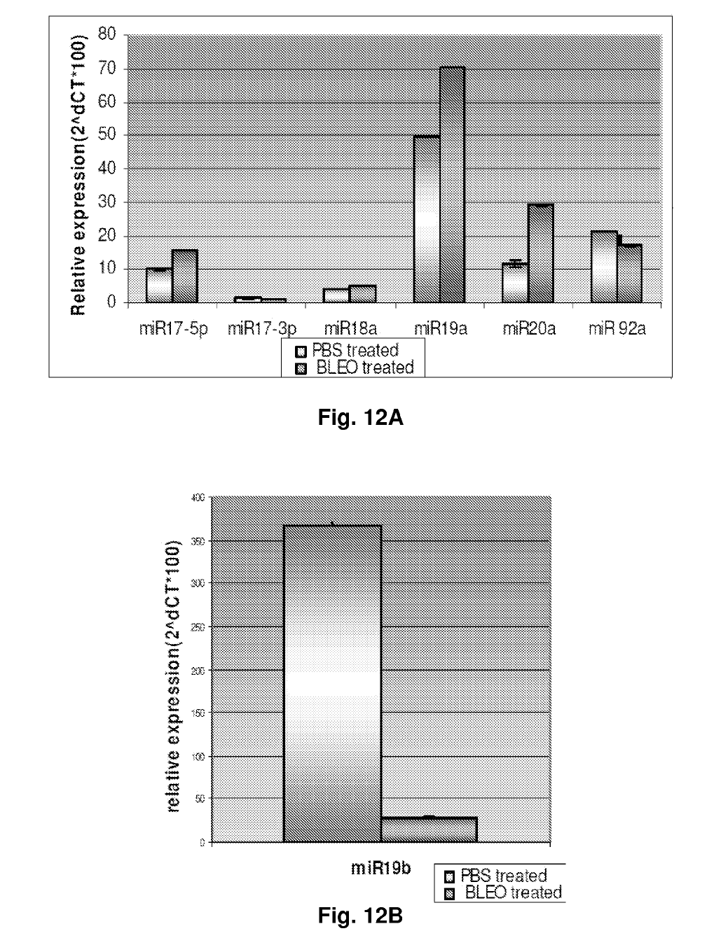

FIG. 11A-1 FIG. 11C are graphs showing the decreased expression of the miR-17˜92 cluster in lung tissue from FVBM mice treated with bleomycin.

FIGS. 11D-11E show the pathological and protein assessment of bleomycin-induced fibrosis in mice. FIG. 11D shows the trichrome staining confirmed collagen deposition in the lungs of mice treated with bleomycin. Shown is a representative image. FIG. 11E shows that a Western blot analysis was performed to examine CTGF, c-myc, phosphorylated c-myc Ets2 and HDAC4. Shown are representative data from seven mice per treatment group.

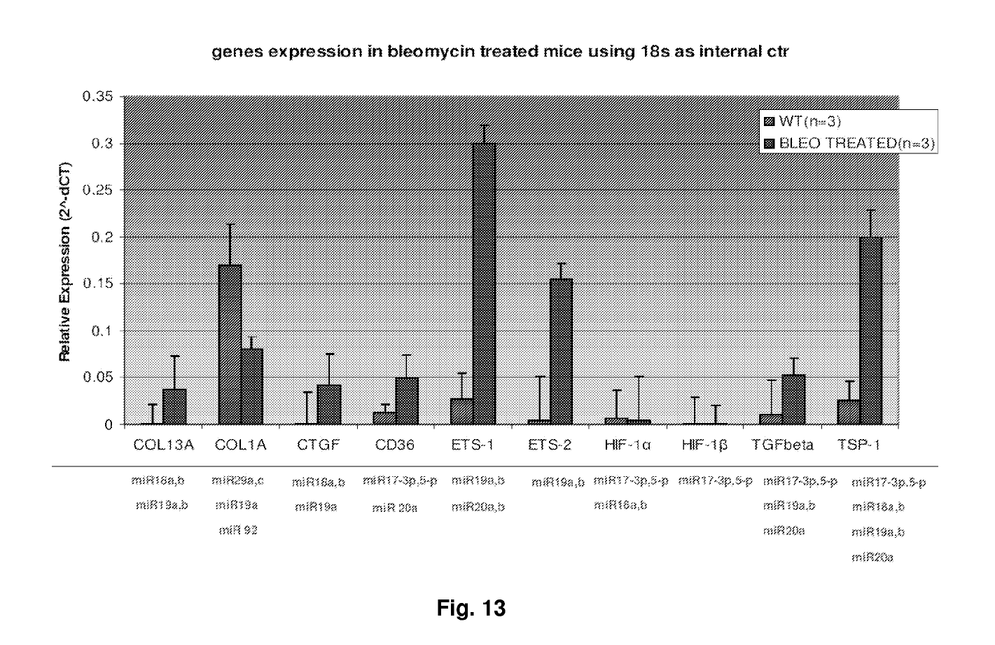

FIGS. 12A-FIG. 12B are graphs showing changes in expression of the miR-17˜92 cluster in bleomycin-induced fibrosis in C57BL/6 mice. FIG. 12A shows the changes in expression of the miR 17˜92 cluster in bleomycin-induced fibrosis in mice. RNA isolated from the lungs of mice was subjected to qRT-PCR using specific primers to each microRNA in the cluster. Relative expression was determined using 18 s RNA as an internal control. Data shown are the average±SEM (n=8). FIG. 13 is a graph showing IPF gene expression in bleomycin treated C67BL/6 mice using 18 s as an internal control.

### Example 4

### Gene Expression in Human Lung Fibroblasts

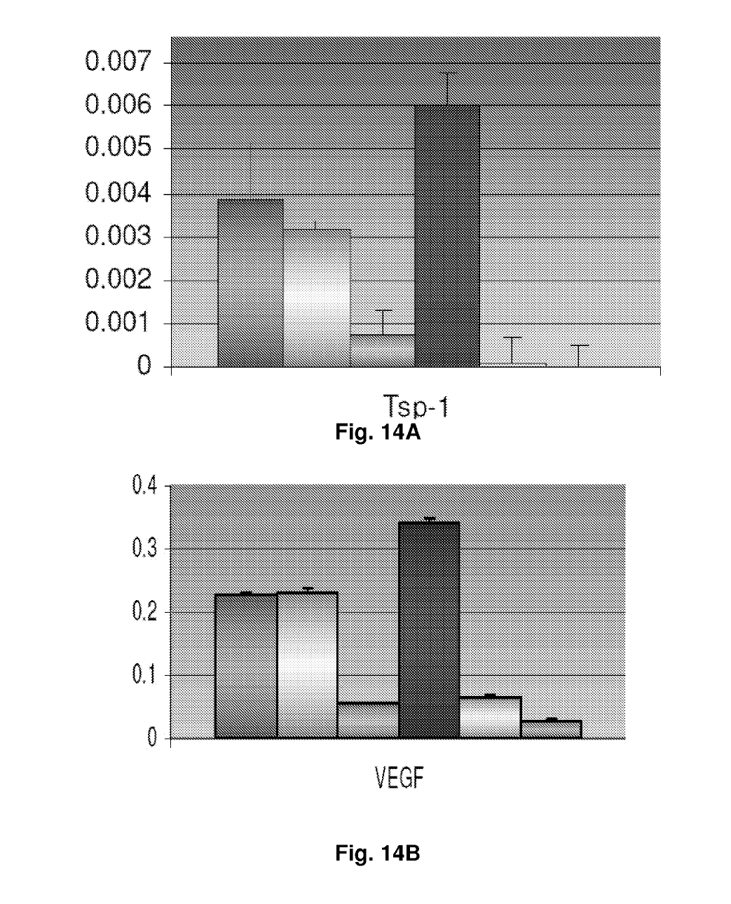

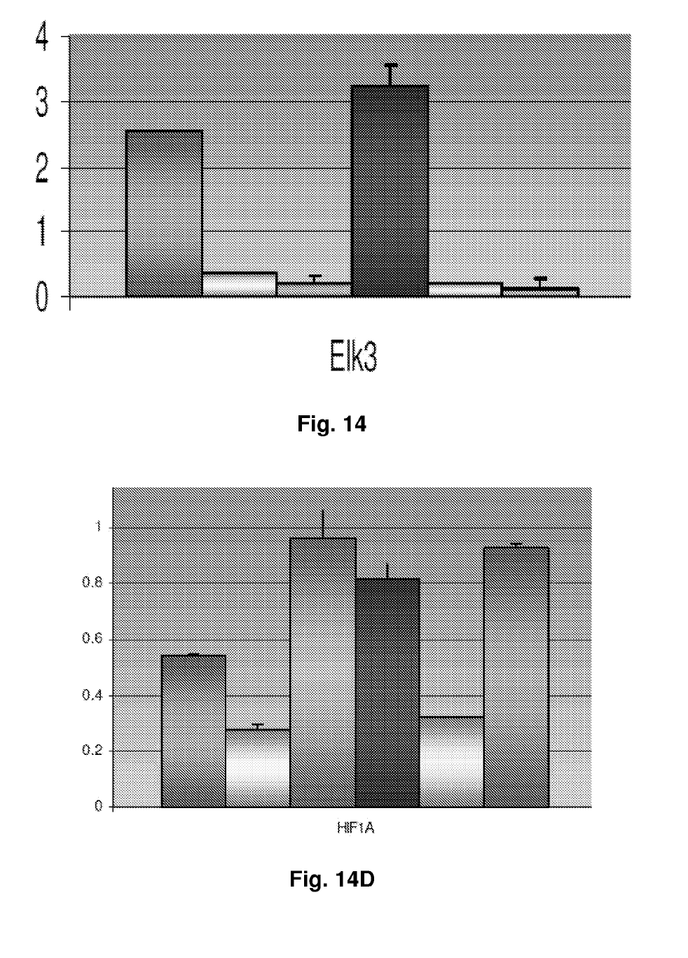

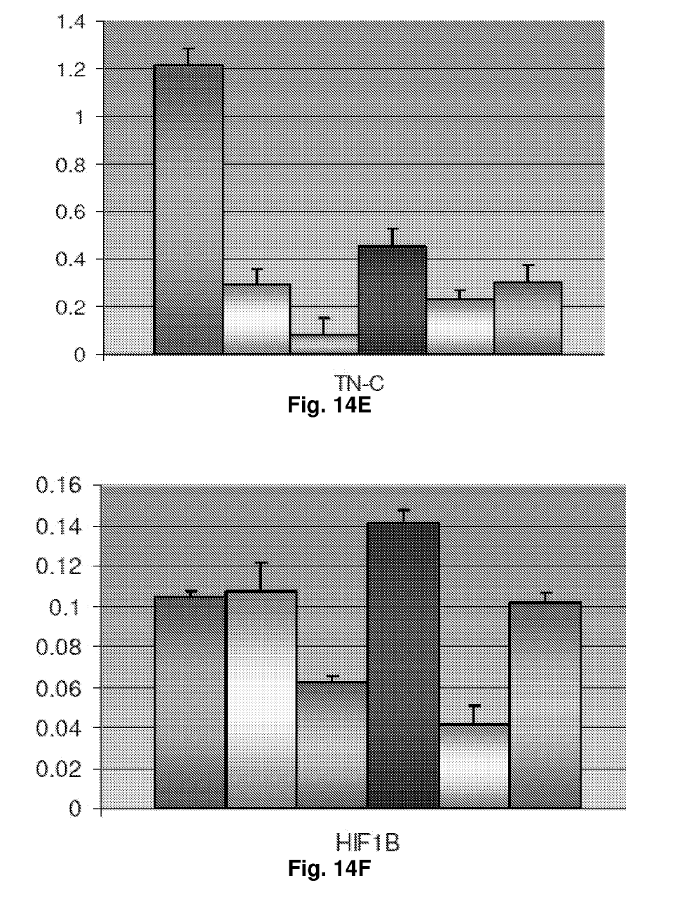

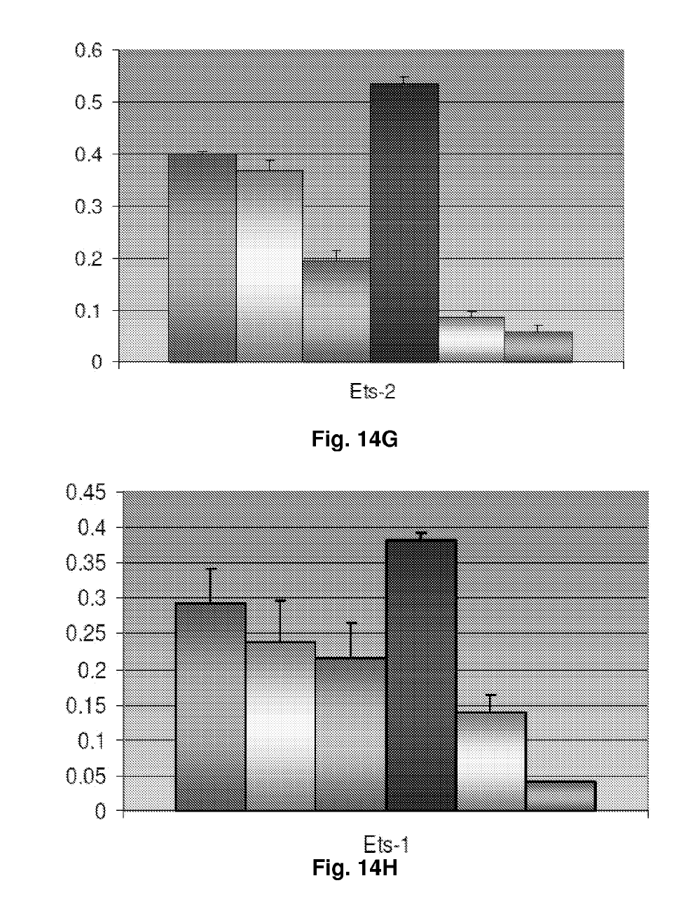

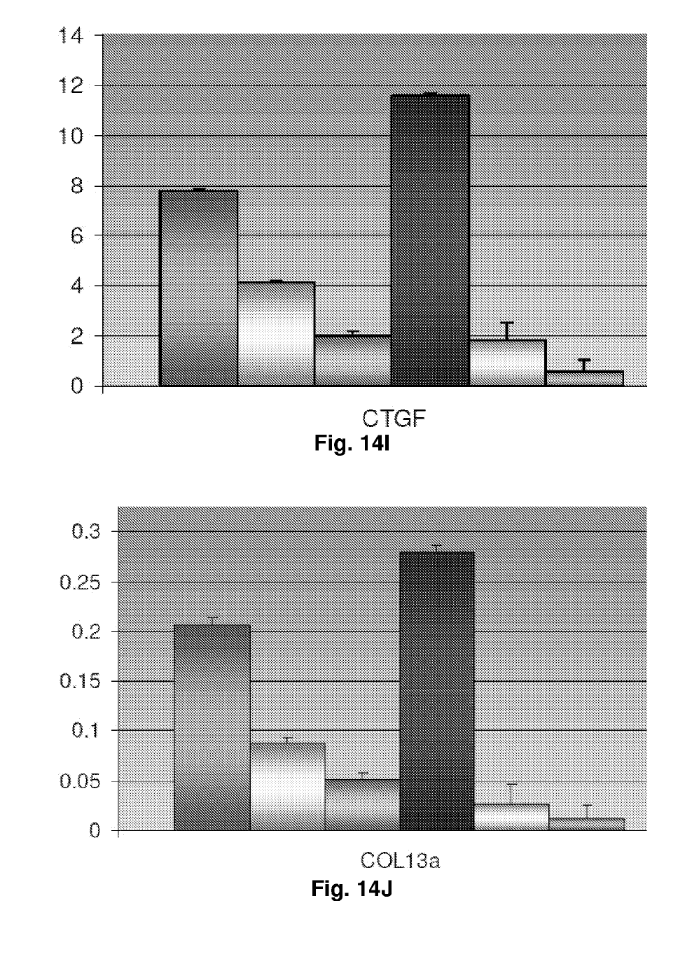

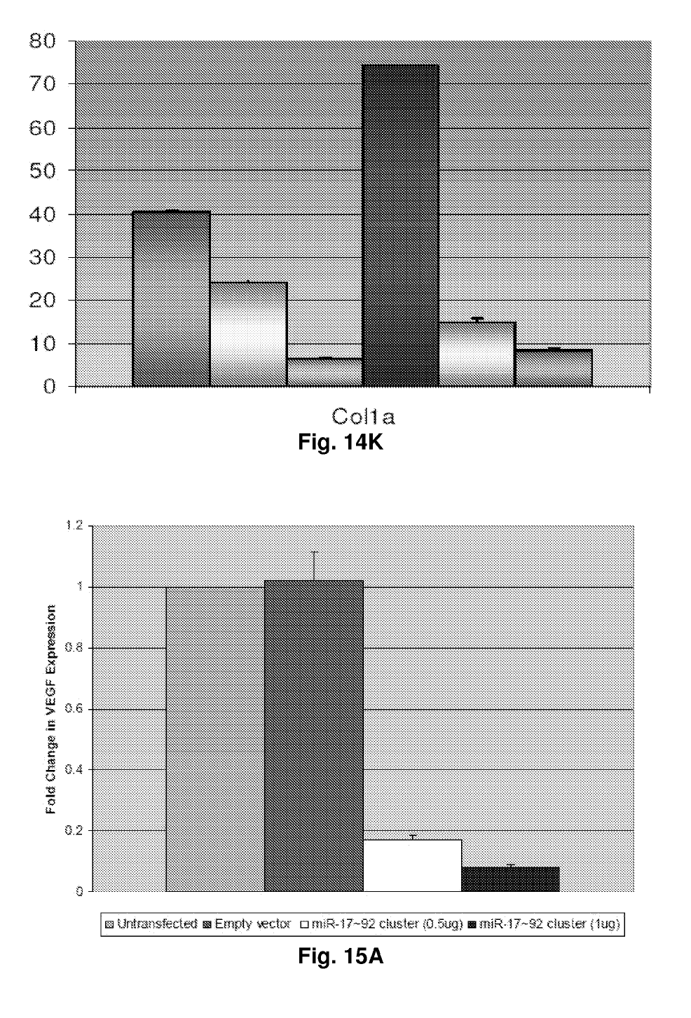

FIGS. 14A-14K are graphs showing the effect of over-expression of miR-17˜92 cluster on IPF gene expression for Tsp-1, VEGF, Elk3, HIF1A, TN—C, HIF1B, Ets-2, Ets-1, CTGF, COL13a and Col1a.

The mean gene expression from the IPF/ILD profiles was calculated, and these values were divided by the mean expression observed in the control samples. These values were used to identify key biological pathways that are likely to be active in the IPF/ILD patients. Ingenuity software analysis scored ten pathways with acceptable P-values of 10⁻² (FIG. 10E).

The inventors identified genes facilitating myofibroblast proliferation, extracellular matrix synthesis, developmental pathways, and angiogenesic gene expression. Genes implicated in these pathways strongly support mesenchymal cell activation and proliferation, but do not allow discrimination among the proposed origins of the regulation of this (myo)fibroblast activity; recruitment of fibroblasts/fibrocytes from the circulation, or the presence of Epithelial cell to Mesenchymal cell Transition (EMT). Other active genes such as VEGF and Notch signaling are consistent with active or aberrant developmental programs, angiogenenic programs and endothelial cell targeting and turnover (Cosgrove et al., 2004; Magro et al., 2006). The genes responsible for triggering the “hepatic fibrosis/stellate cell activation’ pathway emphasize the importance of TGF-β, TGF-α, EGF, and endothelin signaling in the IPF/ILD samples. These signaling molecules in turn regulate many of the effectors of extracellular matrix remodeling including type I and type III collagen, and matrix metalloproteinase-2 and -7.

The samples profiled for mRNA were also profiled for miRNA by a RT-PCR based method. Similar to mRNA profiles, the miRNA profile from lung tissue of patients with IPF/ILD clustered to the right of the figure, while the control profiles grouped to the left, suggesting an emergent miRNA signature or profile in IPF/ILD lung samples. This analysis demonstrates the ability to capture miRNA profiles from frozen samples, stratify the data, and relate the miRNA profiles to mRNA profiles. Hierarchical analysis of the IPF/ILD data by FVC functional group suggests emergence of specific miRNA profiles.

MiR-019b, miR-020a, and miR-106b are highly expressed in control lung tissue, but are markedly reduced in lung tissue from patients with IPF/ILD. These miRNA profiles implicate the miR-17˜92 cluster as a novel target that is reduced in patients with IPF/ILD. Reduced expression of this miRNA cluster may be used to enhance expression of gene networks targeted by these miRNAs.

### Example 5

### Re-Introduction of miRs

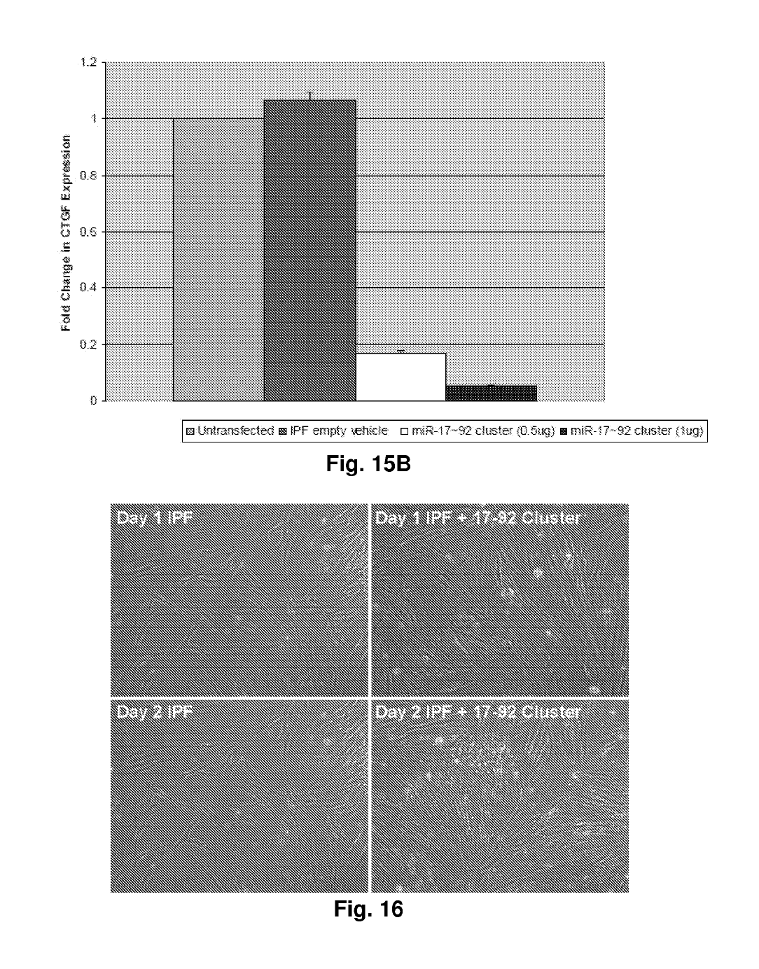

Since the cluster is decreased in the IPF-derived lung fibroblasts and several of the miRNAs contained in the cluster target genes like CTGF and VEGF, the inventors herein determined next examined whether re-introduction of the miR-17˜92 cluster in the IPF cell line decreases the expression of these gene targets. The initial enhanced expression of VEGF and CTGF were markedly reduced by reintroduction of the miR-17˜92 cluster in fibroblasts derived from IPF patients lungs (FIG. 15A-FIG. 15B). This demonstrates that these findings are not patient or cell line specific. Also, distinct phenotypic changes in cells transfected with the cluster compared untransfected cells were found, as shown in FIG. 16, which shows that the fibroblasts appeared to organize in a contiguous cell sheet.

Using two methods of detection the microarray chip and qRT-PCR, the inventors herein found many differences in the miRNA expression. MgiR-19b was consistently decreased between the two methods. A high similarity in expression of miR-17˜92 cluster was found between human and mouse. While not wishing to be bound by theory, the inventors herein now believe that increases in CTGF protein in IPF are most likely due to decreases in expression of the miR-19b from the miR-17˜92 cluster. This observation was seen in both human and mouse samples. In addition, miR-19b and miR-20a are down regulated with increasing severity of disease in patients with IPF.

### Example 6

### Targeted Genes

Several genes were identified that are targeted by the miR-17˜92 cluster. The expression of these genes as well as corresponding protein lung tissue based on disease severity were examined.

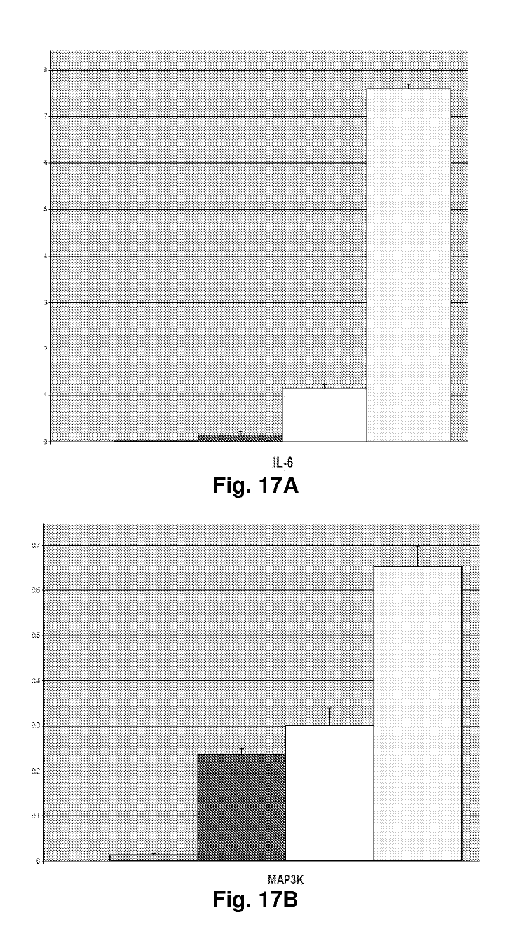

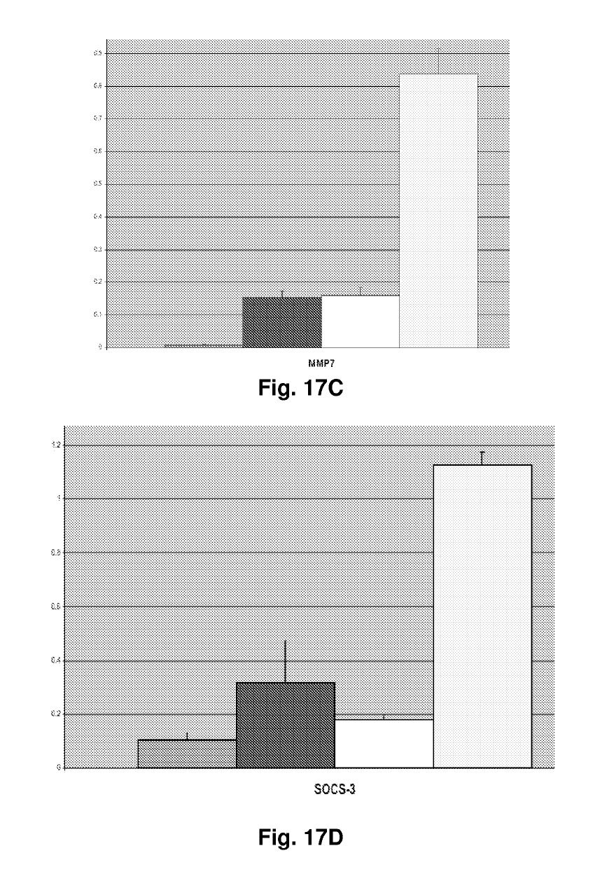

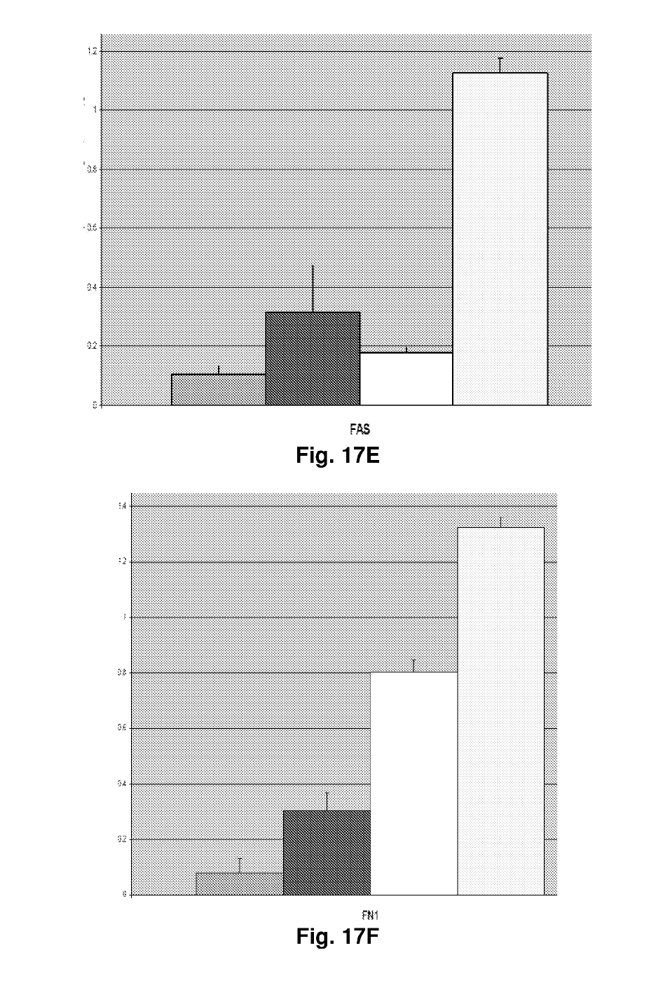

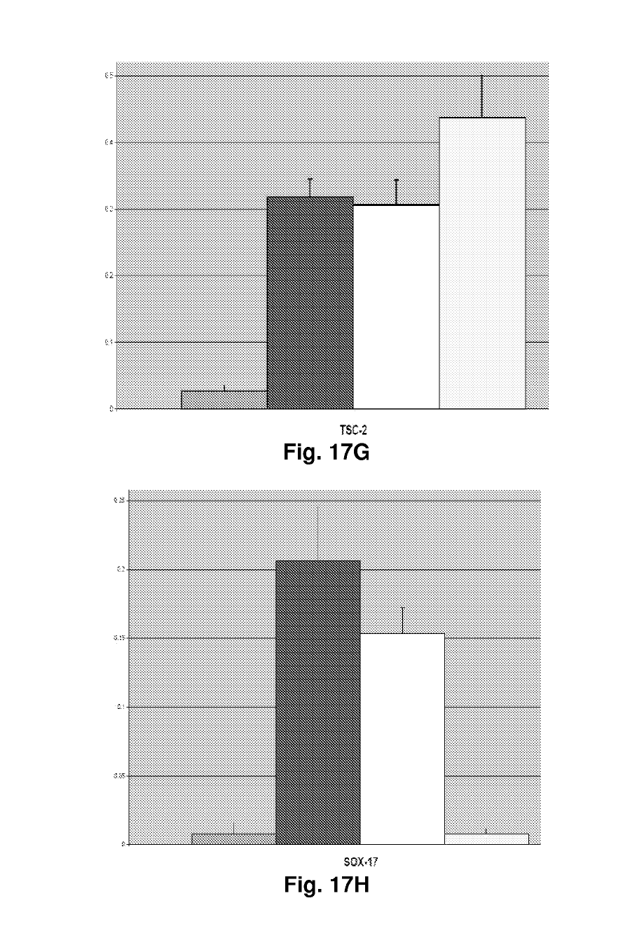

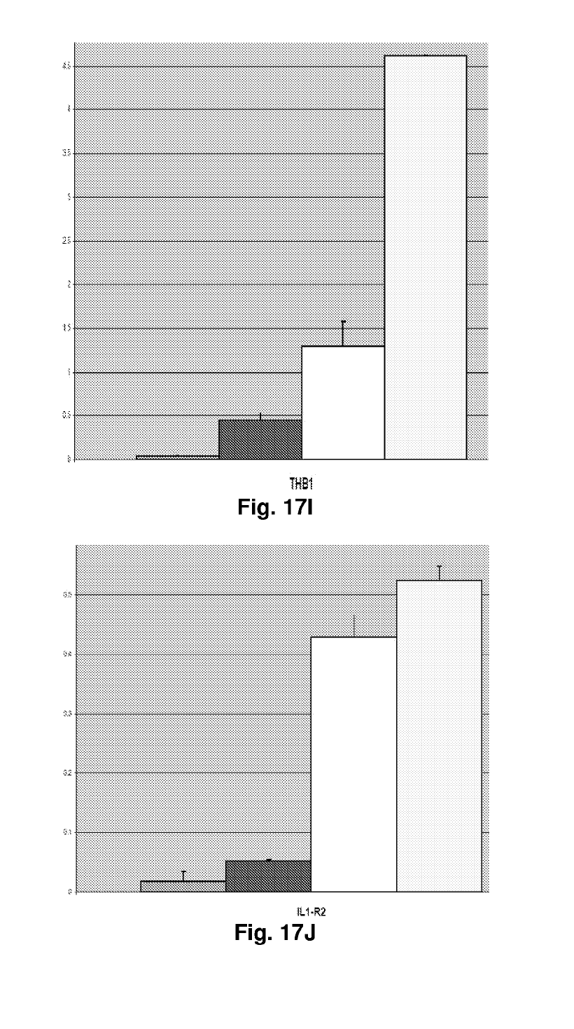

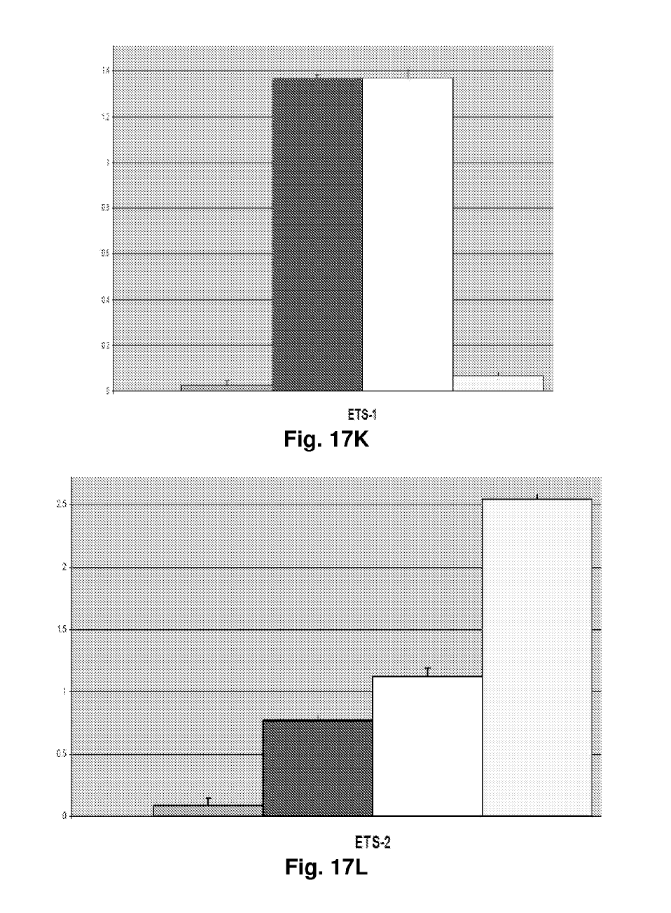

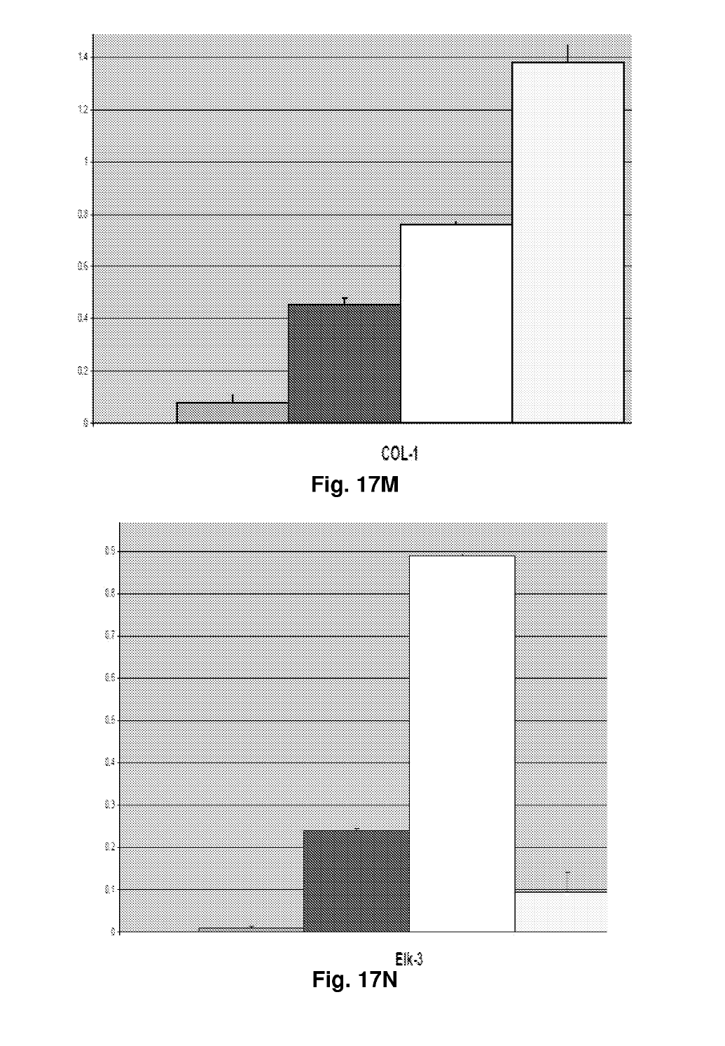

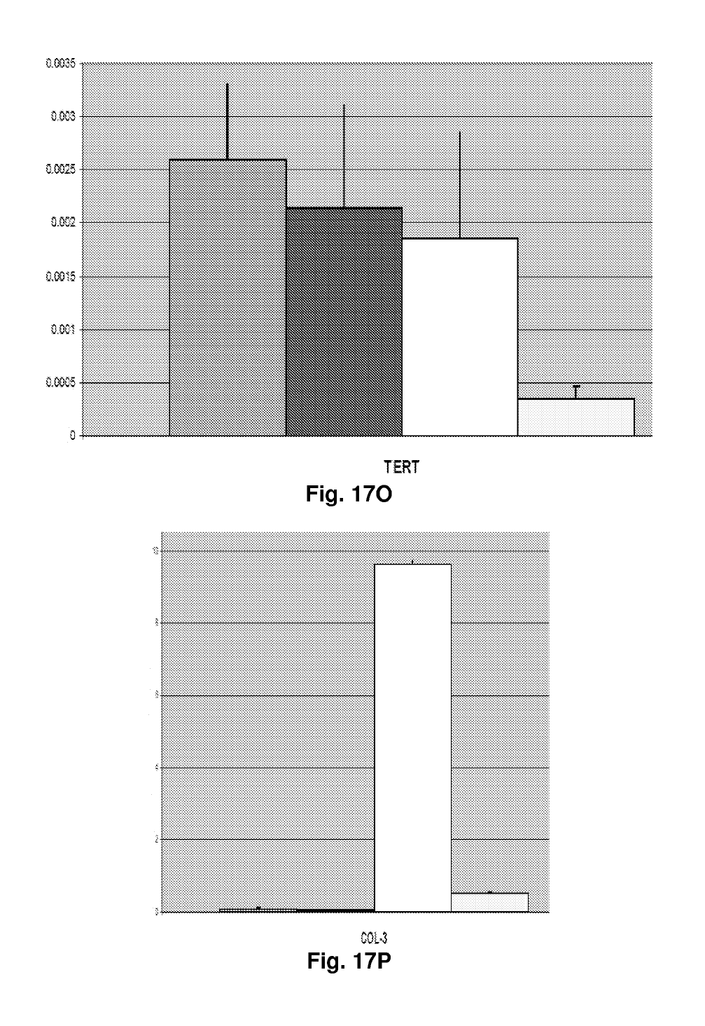

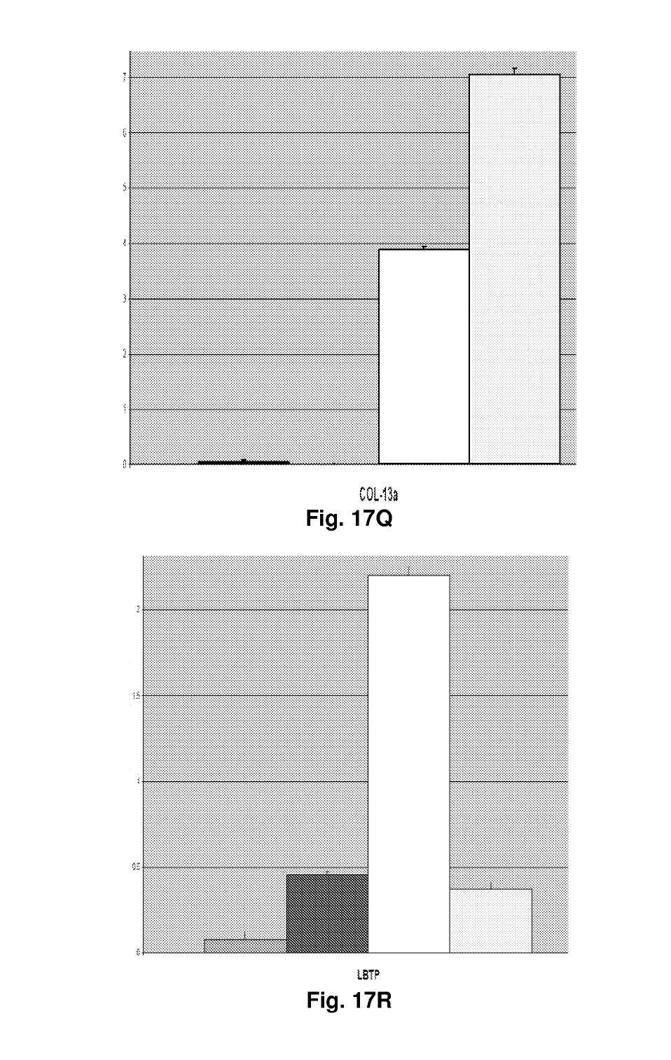

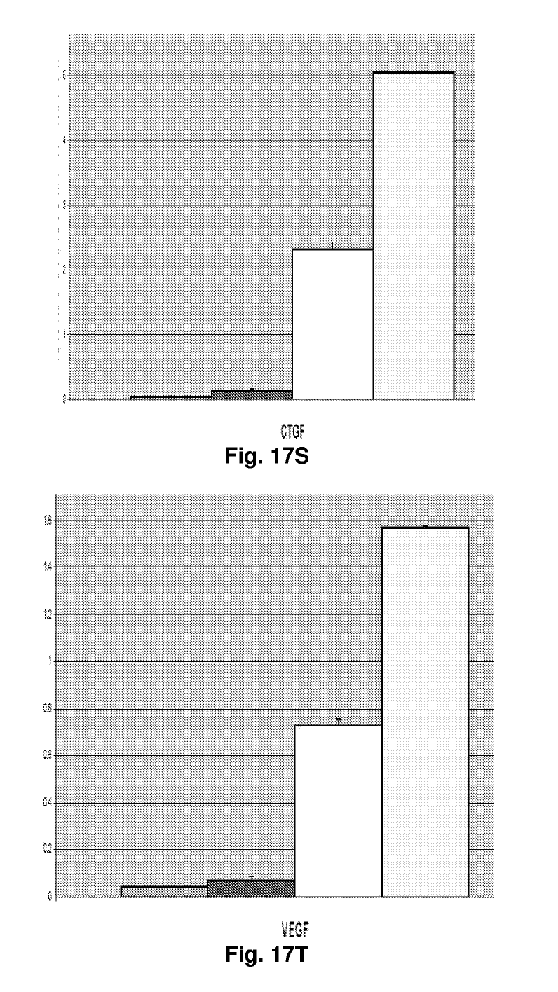

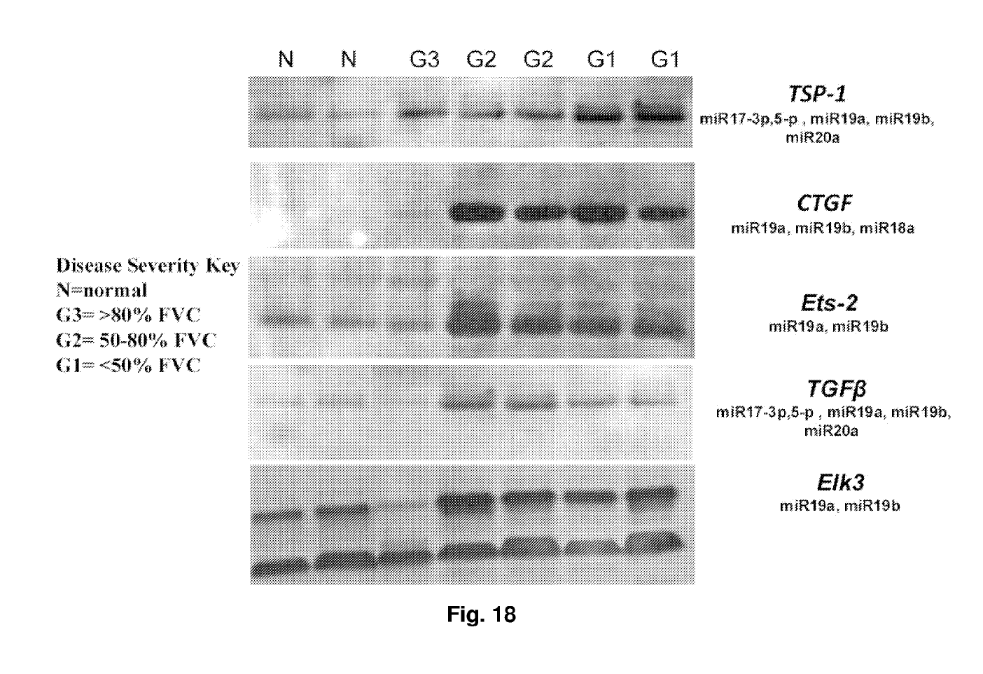

FIGS. 17A-17T show graphs for gene expression in patient samples based on disease severity. FIG. 18 shows protein expression in lung tissue from patients with IPF.

In situ Hybridization

In situ hybridization was conducted to confirm qRT-PCR analysis that expression of miR-19b and -20a are decreased in lung tissue from patients with IPF compared to normal tissue.

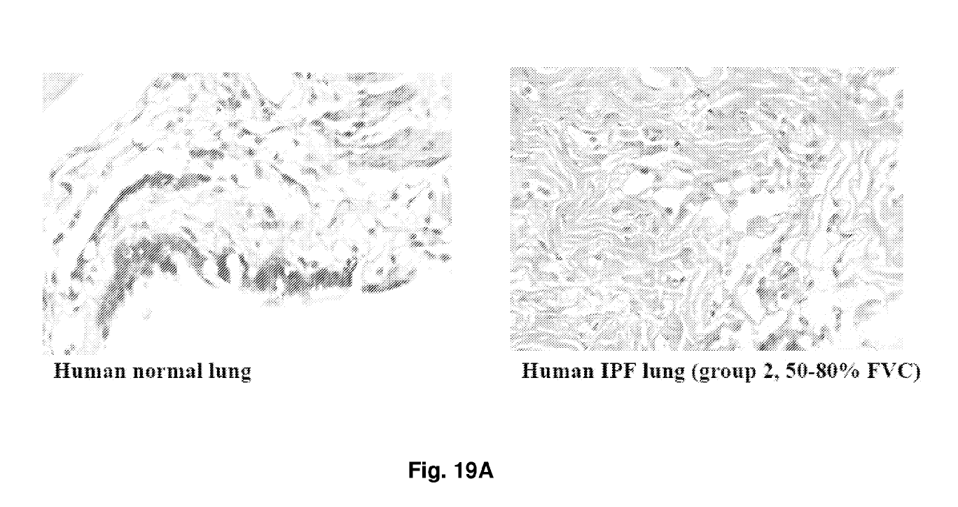

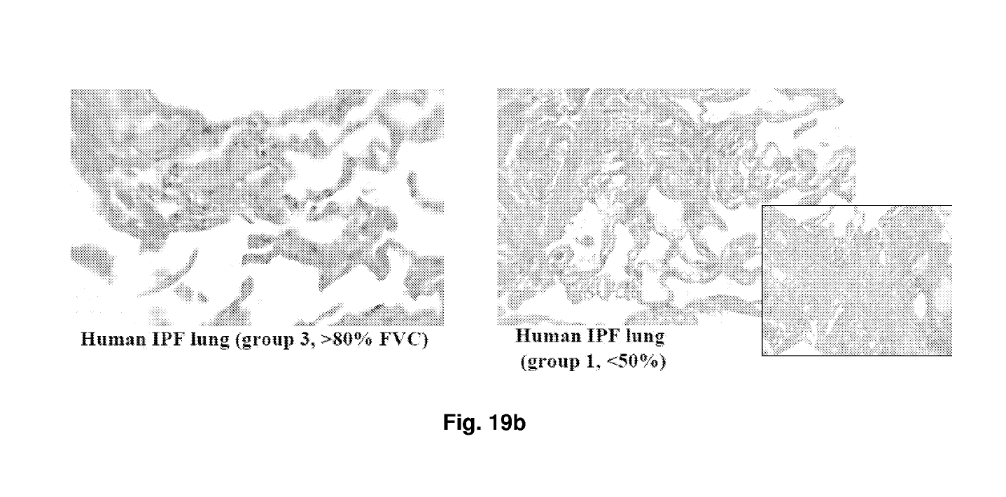

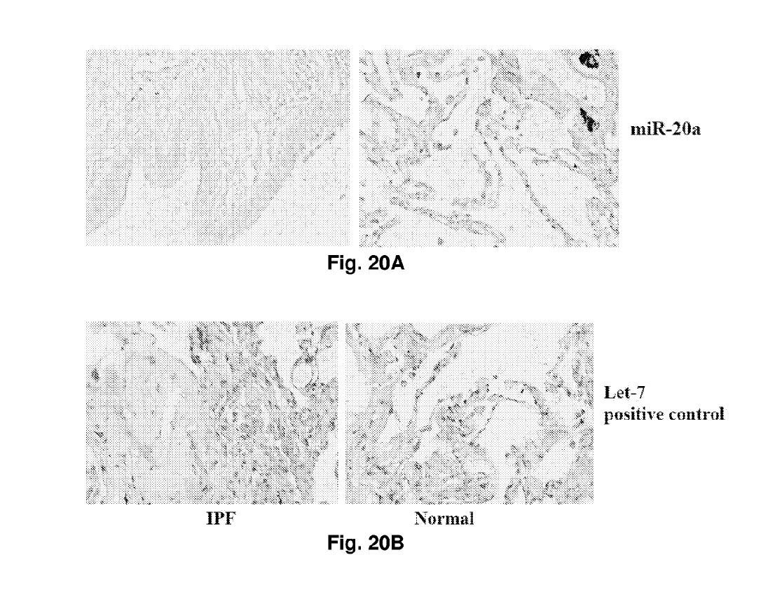

FIGS. 19A-19B show the miR-19b expression in human lung tissue.

FIGS. 20A-20B show the miR-20a and Let-7 expression in human lung tissue.

Expression in Cell Lines

Decrease expression of the miR-17˜92 cluster in lung fibroblast cell lines derived from patients IPF compared to normal human lung fibroblast cell lines.

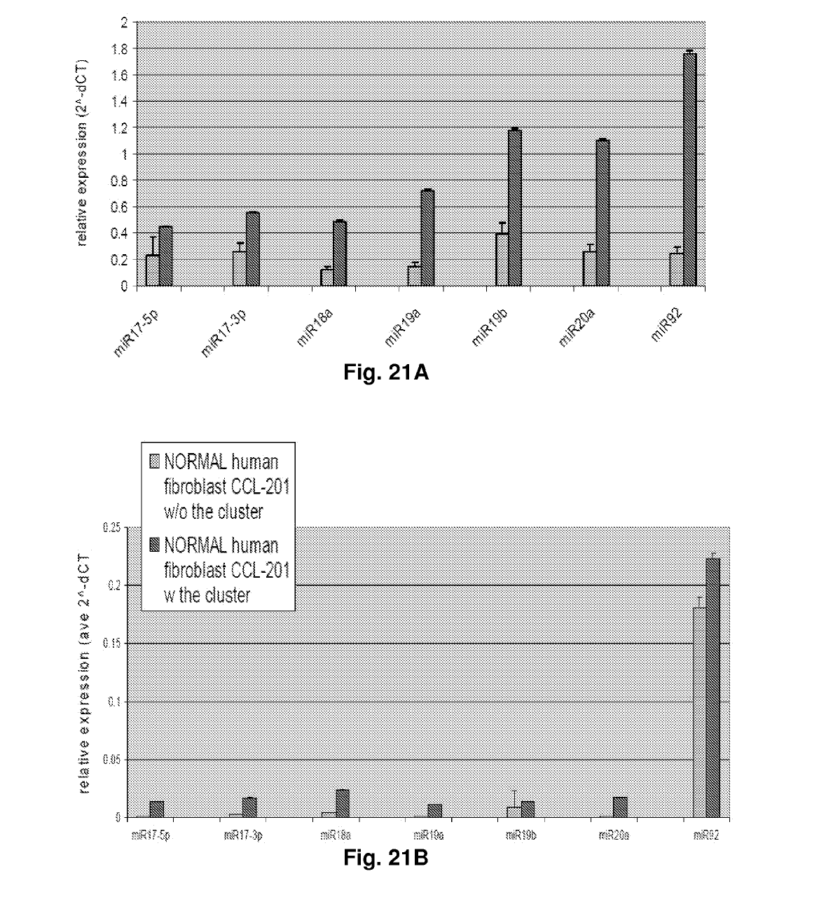

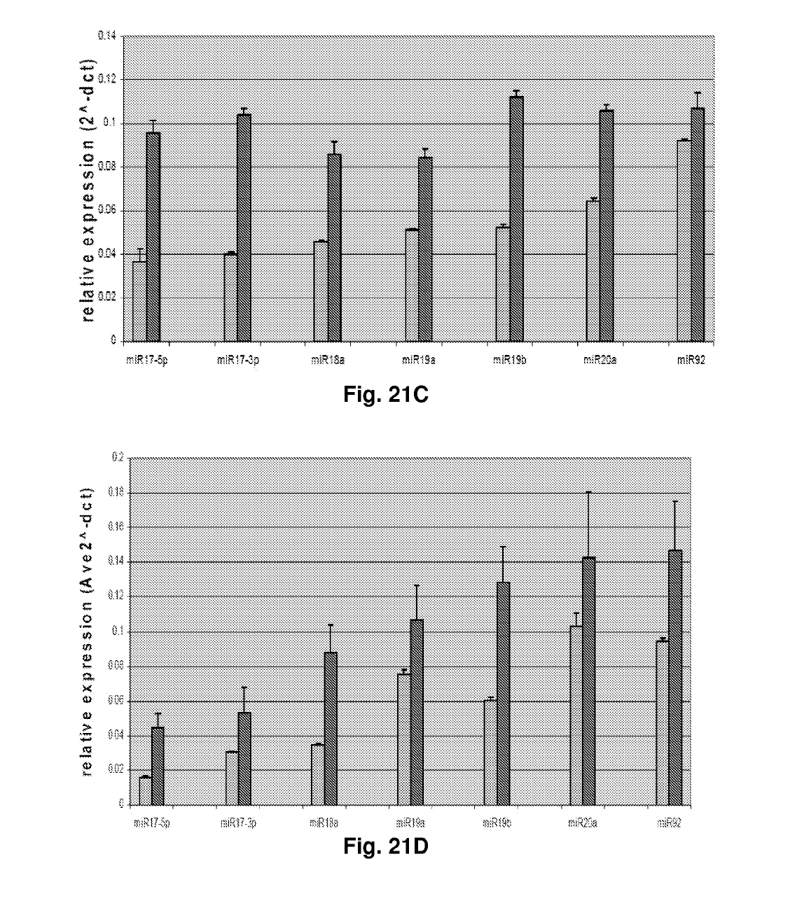

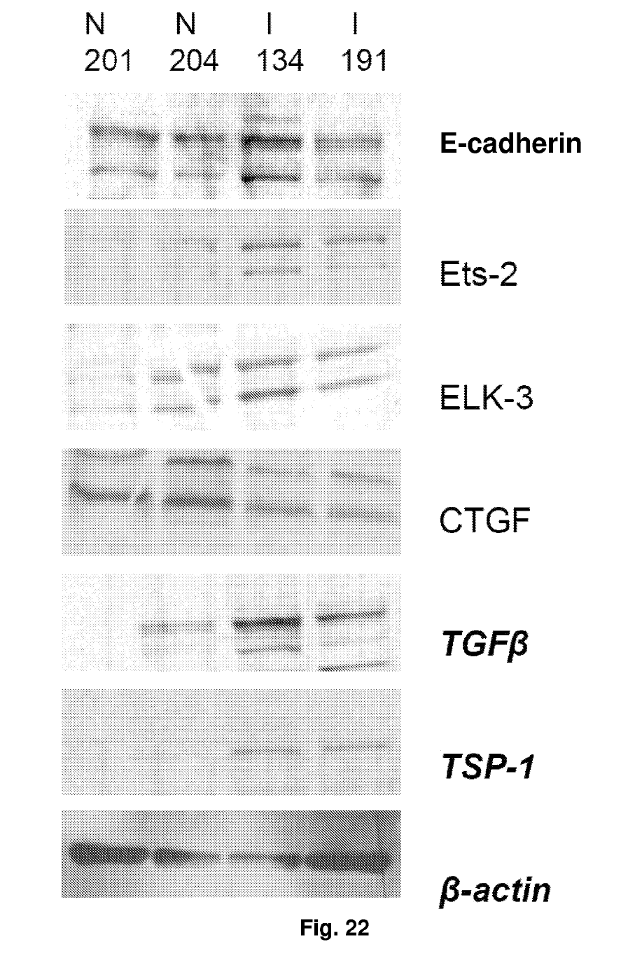

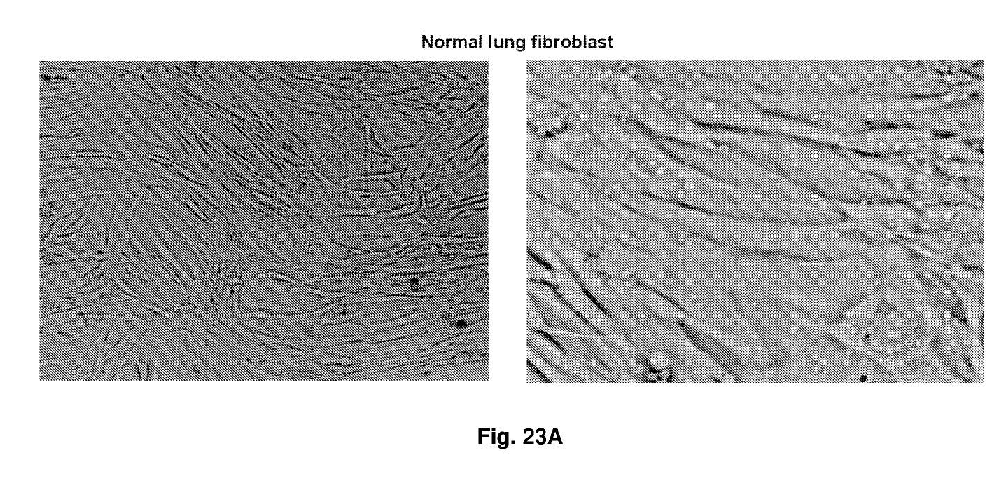

FIGS. 21A-21D are graphs showing the miRNA expression in human lung fibroblast cell lines. Protein expression in human normal and IPF lung fibroblast cell lines is shown in FIG. 22, where N=Normal, and I=IPF.

### Example 7

### Phenotypic Differences

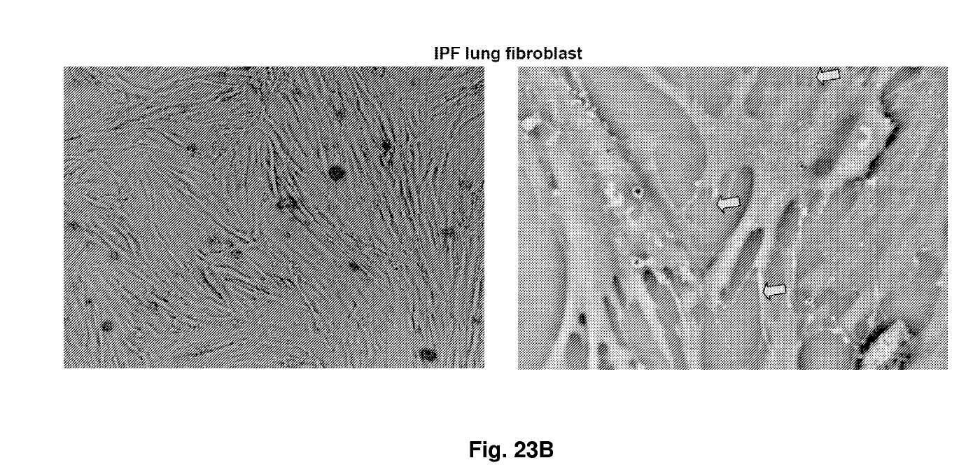

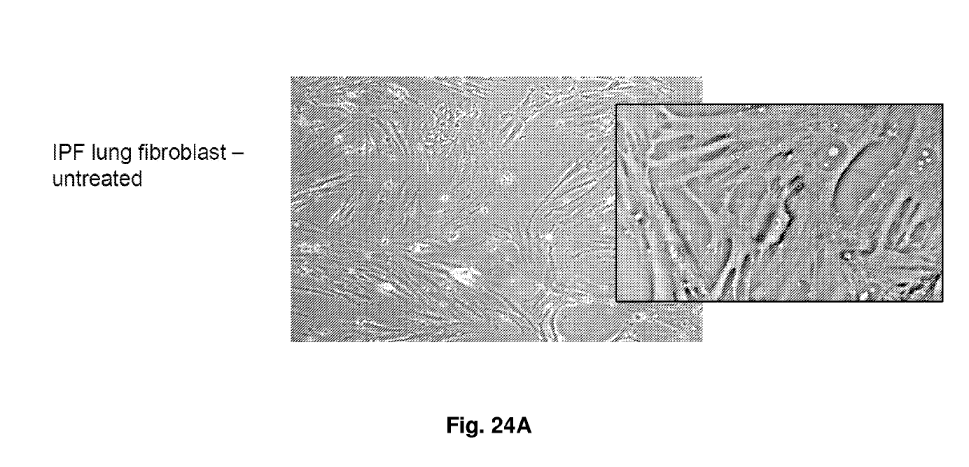

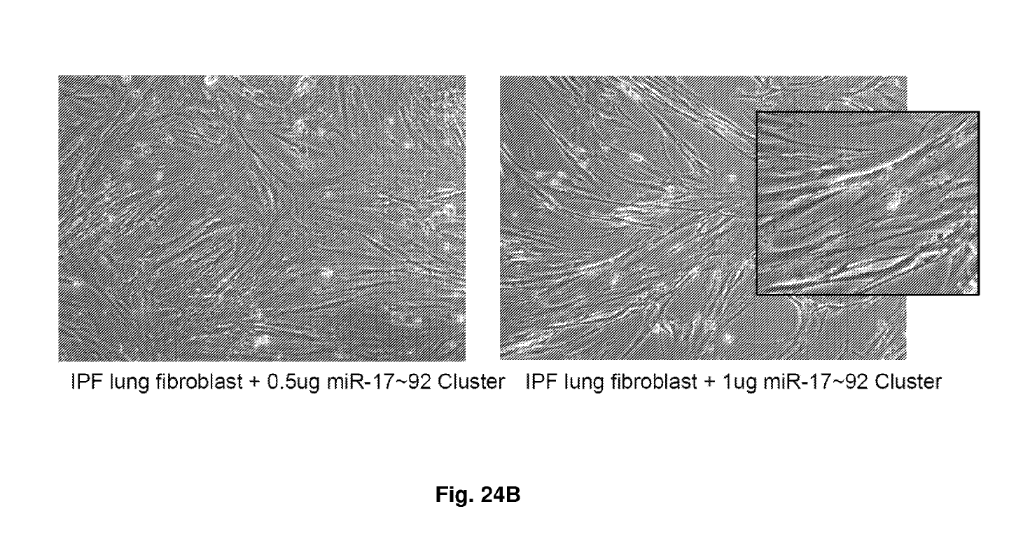

IPF-derived lung fibroblasts appear phenotypically different with more filipodia compared to normal human lung fibroblast cell lines. FIGS. 23A-23B show the morphology for human lung fibroblasts in Normal and IPF.

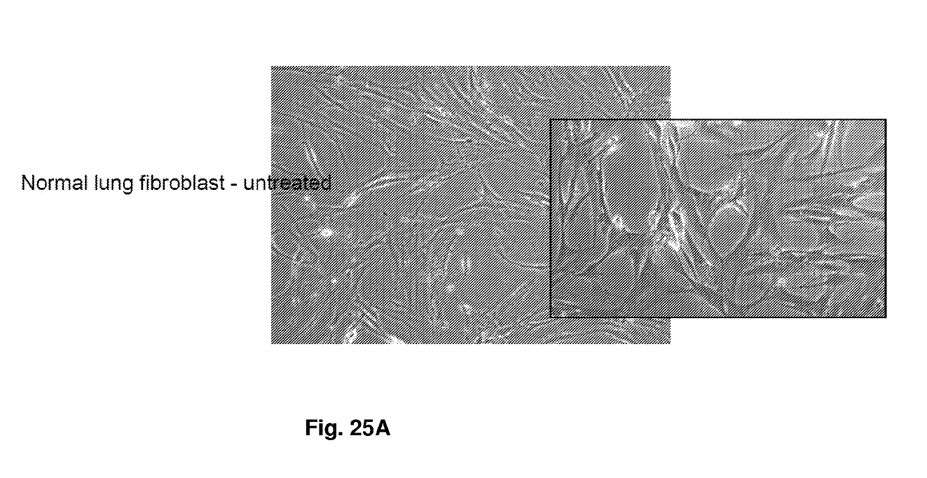

Since the miR-17˜92 cluster, as well as miR-19b and miR-20a, are decreased in the IPF-derived fibroblasts similar to tissue from patients with IPF, the inventors manipulated their expression in vitro to examine phenotypic and molecular changes. FIGS. 24A-24B show IPF-derived fibroblasts transfected with the miR-17˜92 cluster begin to assume a phenotype similar to normal lung fibroblasts.

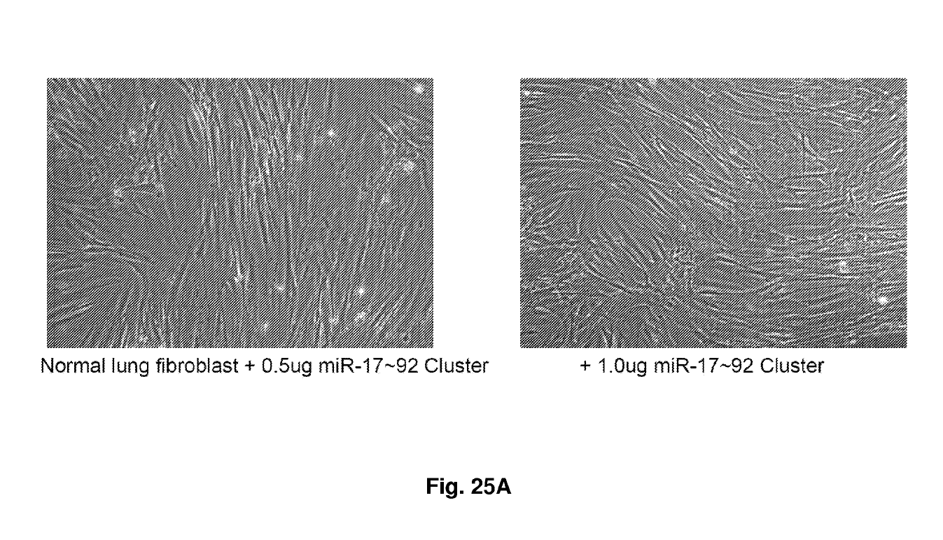

Overexpression of the miR-17˜92 cluster in normal lung fibroblasts does not alter their phenotype, as shown in FIGS. 25A-25B.

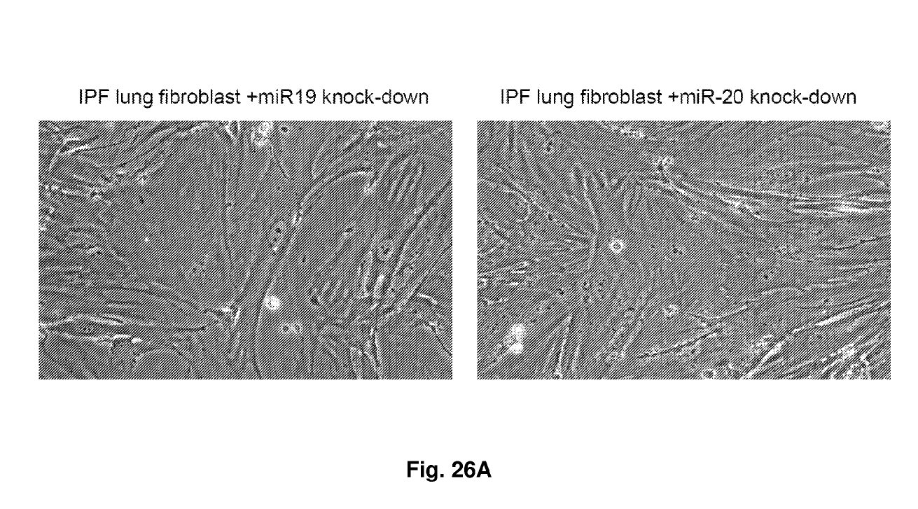

Overexpression but not knockdown expression of miR-19b or miR-20a induced phenotypic changes in IPF lung fibroblast cell lines, as shown in FIGS. 26A-26B.

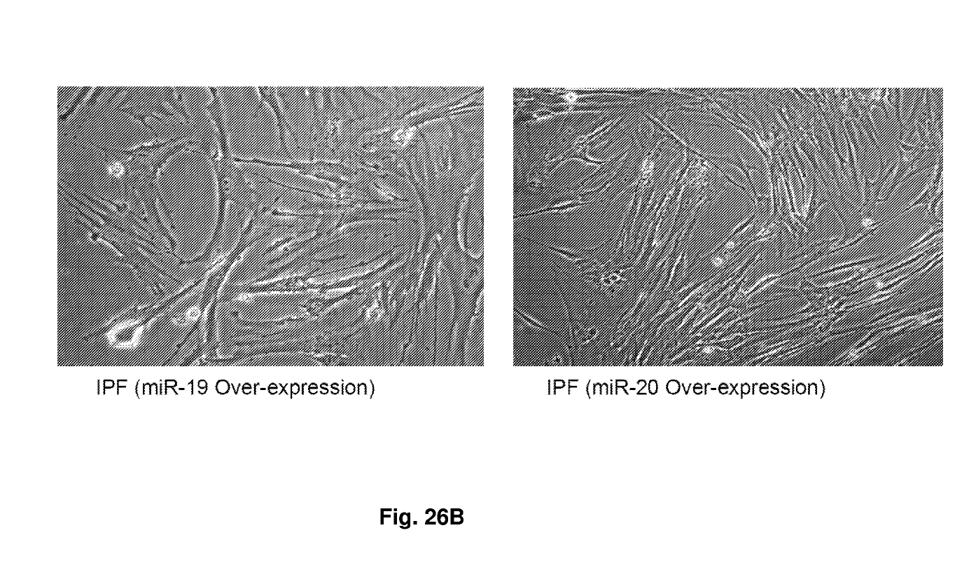

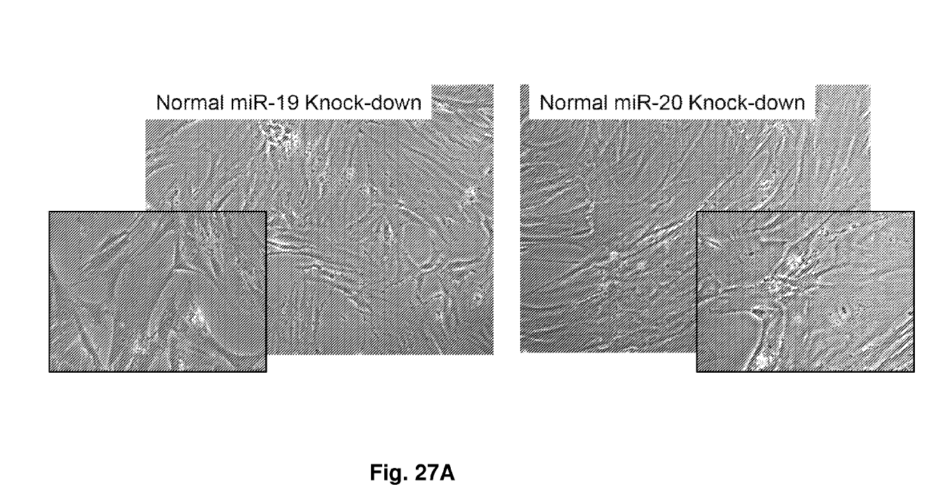

Knockdown expression of miR-19b or miR-20a induced normal lung fibroblast cell lines to become phenotypically similar to the IPF lung fibroblast cell lines, as shown in FIGS. 27A-27B.

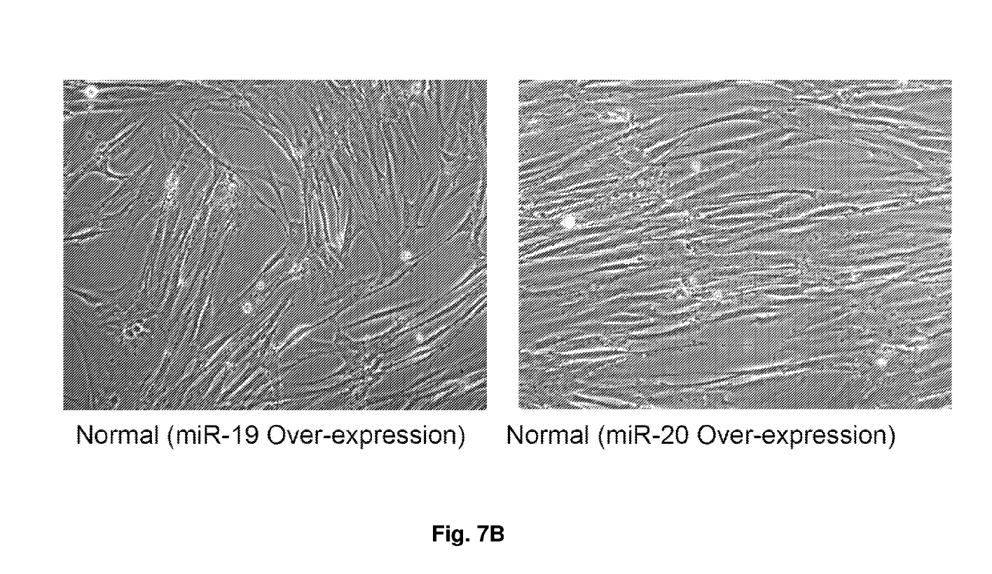

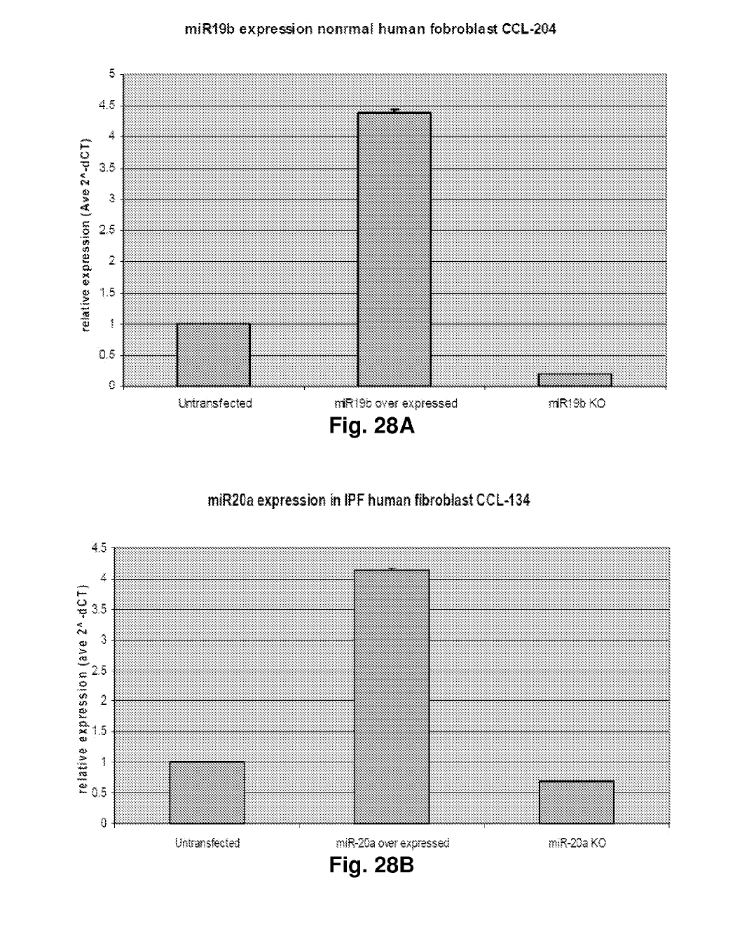

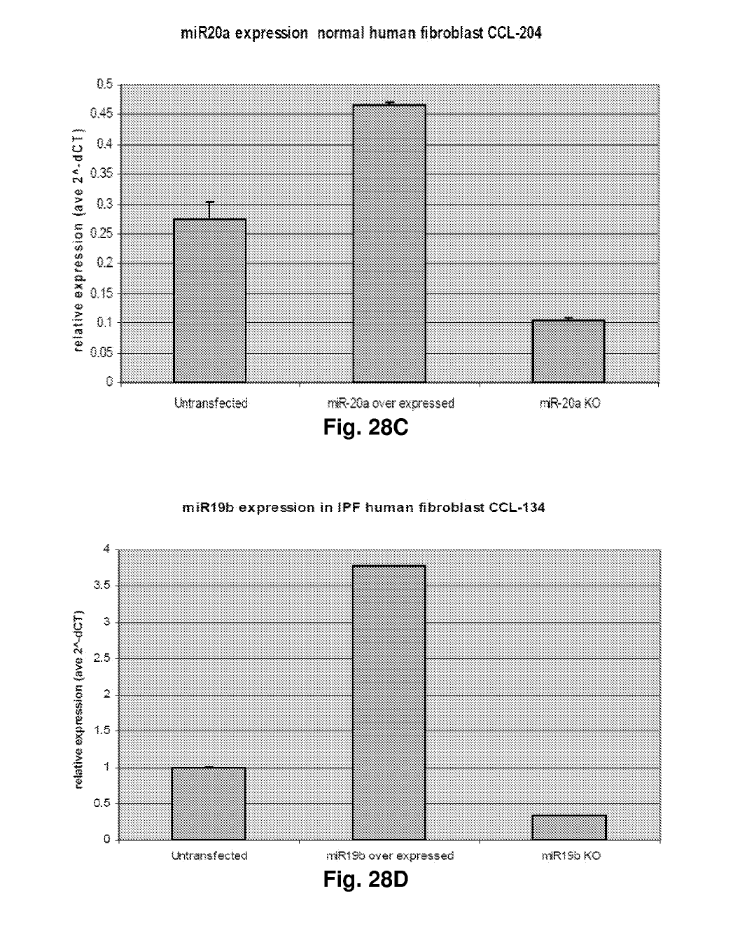

Confirmation of miR-19b and miR-20a expression in human lung fibroblasts after transfections is shown in FIGS. 28A-28D.

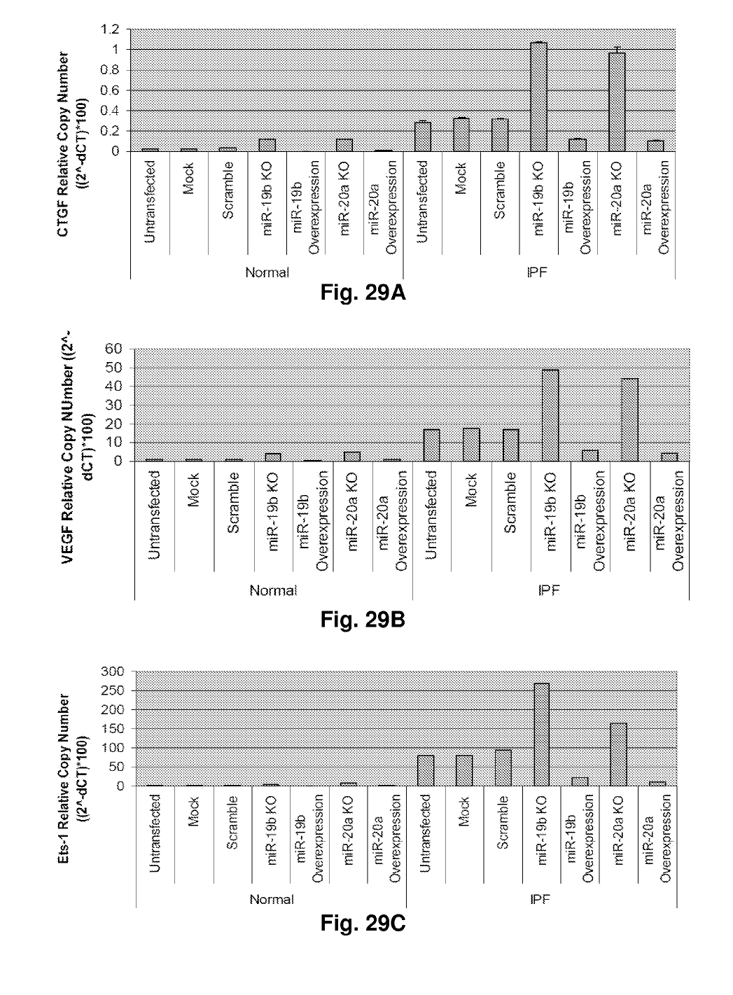

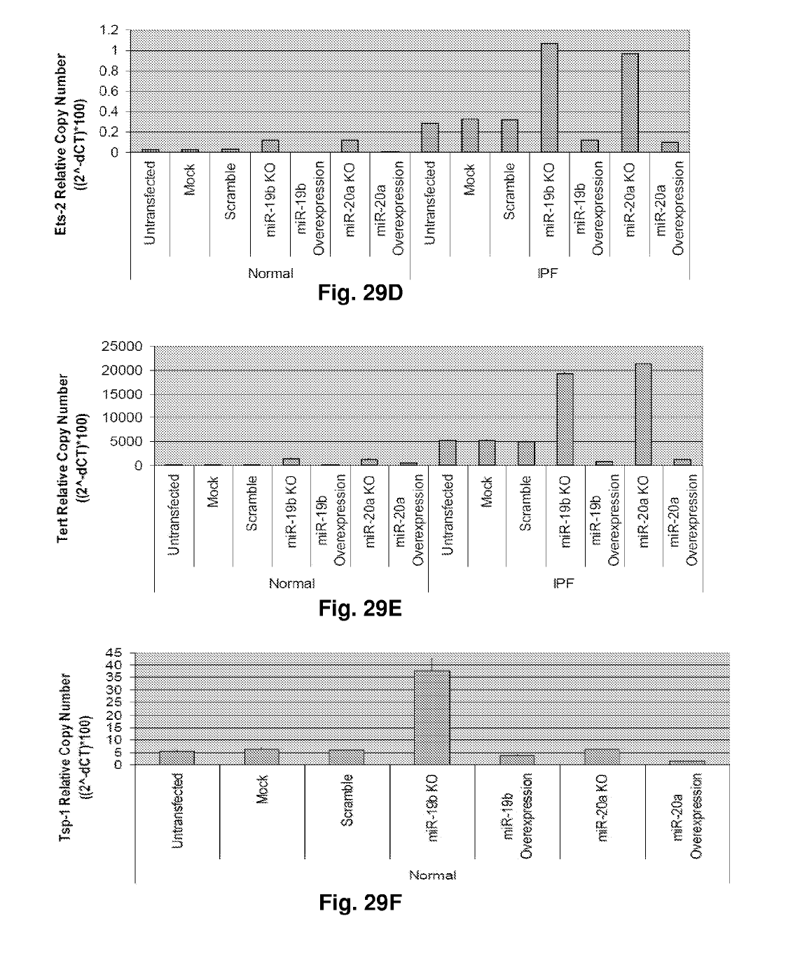

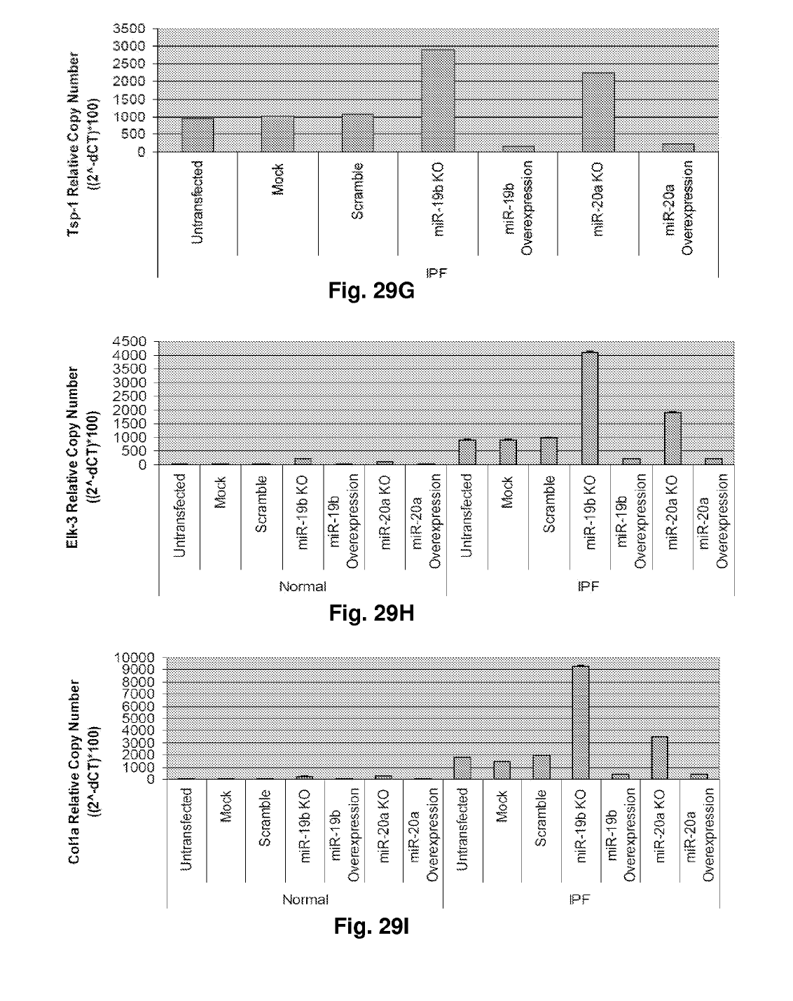

There is an increase gene expression in both normal and IPF fibroblast cell lines when expression of either miR-19b or miR20a is knockdown. In contrast, overexpression of these miRNAs resulted in decrease expression of the targeted genes. FIGS. 29A-29J show changes in gene expression.

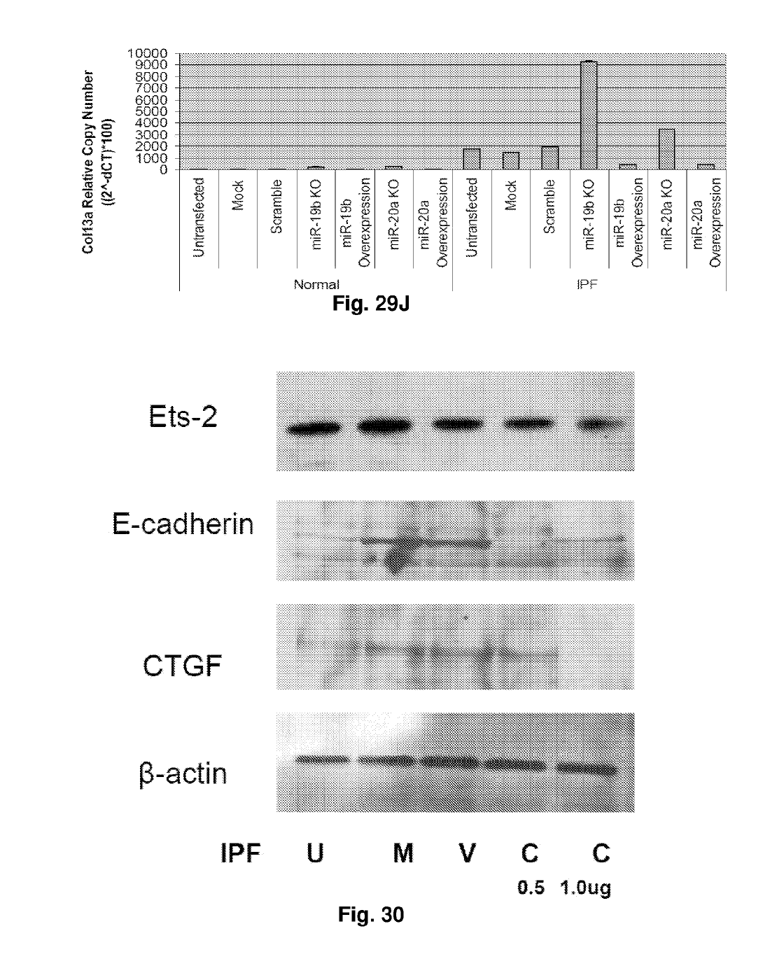

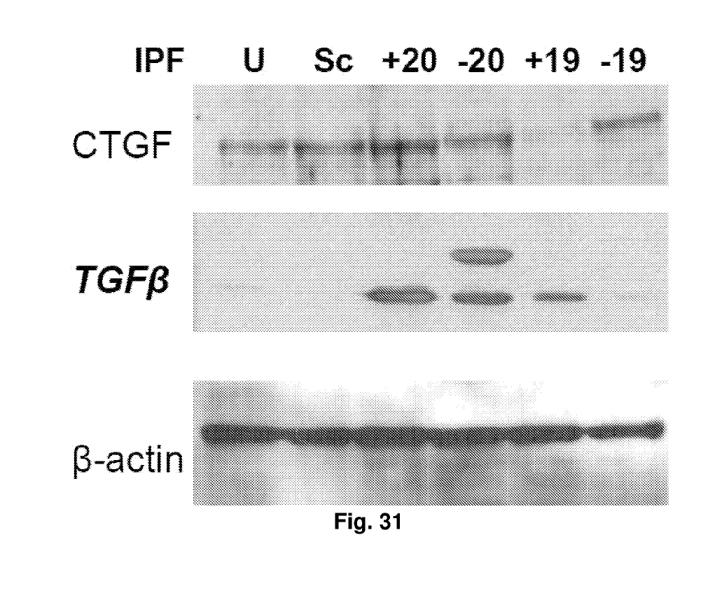

An analysis of protein expression in IPF lung fibroblast cell lines transfected with miR-17˜92 cluster was conducted. Decreased protein expression following transfection of miR-17˜92 in IPF lung fibroblast cell lines was found, as shown in FIG. 30, where U=Untransfected, M=Mock transfection, V=Empty vector and C=miR-17˜92 cluster.

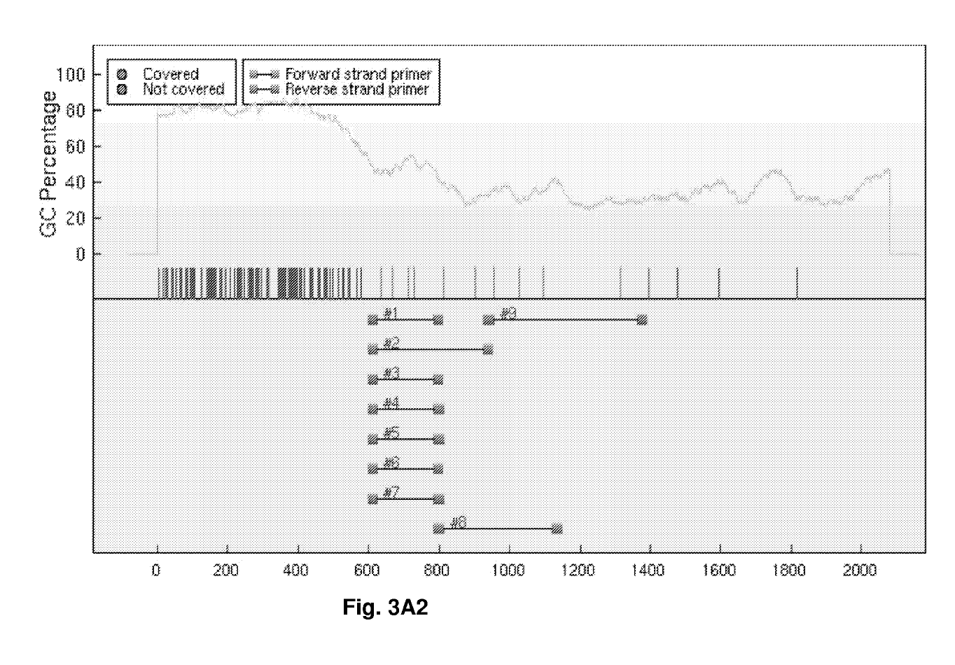

Decreased protein expression following transfection of either miR-19b or miR-20a in IPF lung fibroblast cell lines is shown in FIG. 31, where U=untransfected, Sc=scramble control, +20=miR-20a, 20=miR-20a antagomirs, +19=miR-19b, and 19=miR-19b antagomirs.

Since the promoter of the miR-17˜92 cluster is rich in CpG islands, the inventors herein now believe that the decrease in the expression of the cluster is due to epigenetic changes. The Location of CpG islands in the promoter of miR-17˜92 and primer sequences used for DNA methylation studies are shown in FIGS. 32A-32B.

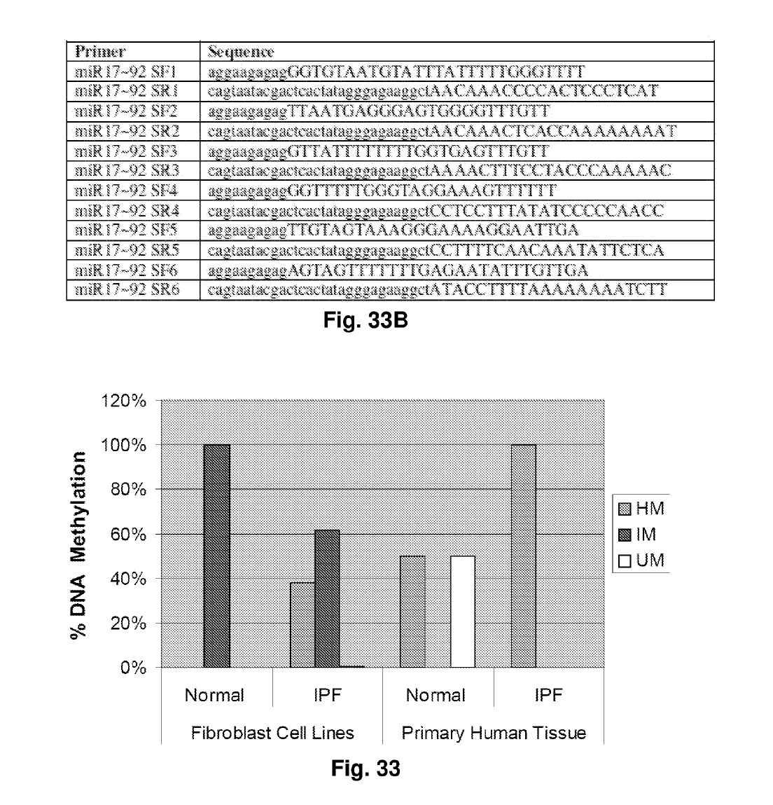

Increased DNA methylation of miR-17˜92 promoter in IPF tissue and fibroblast cell lines compared to normal tissue and cells is shown in FIG. 33.

### EXAMPLES OF USES

As described and exemplified herein particular miRNA are up- or down-regulated during tissue injury and/or inflammation.

As used herein interchangeably, a “miR gene product,” “microRNA,” “miR” or “miRNA” refers to the unprocessed or processed RNA transcript from a miR gene. As the miR gene products are not translated into protein, the term “miR gene products” does not include proteins. The unprocessed miR gene transcript is also called a “miR precursor,” and typically comprises an RNA transcript of about 70-100 nucleotides in length. The miR precursor can be processed by digestion with an RNAse (for example, Dicer, Argonaut, RNAse III (e.g., E. coli RNAse III)) into an active 19-25 nucleotide RNA molecule. This active 19-25 nucleotide RNA molecule is also called the “processed” miR gene transcript or “mature” miRNA.

The active 19-25 nucleotide RNA molecule can be obtained from the miR precursor through natural processing routes (e.g., using intact cells or cell lysates) or by synthetic processing routes (e.g., using isolated processing enzymes, such as isolated Dicer, Argonaut, or RNAse III). It is understood that the active 19-25 nucleotide RNA molecule can also be produced directly by biological or chemical synthesis, without having to be processed from the miR precursor. When a microRNA is referred to herein by name, the name corresponds to both the precursor and mature forms, unless otherwise indicated.

The methods comprise determining the level of at least one miR gene product in a sample from the subject and comparing the level of the miR gene product in the sample to a control. As used herein, a “subject” can be any mammal that has, or is suspected of having, such disorder. In a preferred embodiment, the subject is a human who has, or is suspected of having, such disorder.

The level of at least one miR gene product can be measured in cells of a biological sample obtained from the subject. The sample can be removed from the subject, and DNA can be extracted and isolated by standard techniques. For example, in certain embodiments, the sample can be obtained from the subject prior to initiation of radiotherapy, chemotherapy or other therapeutic treatment. A corresponding control sample, or a control reference sample (e.g., obtained from a population of control samples), can be obtained from unaffected samples of the subject, from a normal human individual or population of normal individuals, or from cultured cells corresponding to the majority of cells in the subject's sample. The control sample can then be processed along with the sample from the subject, so that the levels of miR gene product produced from a given miR gene in cells from the subject's sample can be compared to the corresponding miR gene product levels from cells of the control sample. Alternatively, a reference sample can be obtained and processed separately (e.g., at a different time) from the test sample and the level of a miR gene product produced from a given miR gene in cells from the test sample can be compared to the corresponding miR gene product level from the reference sample.

In one embodiment, the level of the at least one miR gene product in the test sample is greater than the level of the corresponding miR gene product in the control sample (i.e., expression of the miR gene product is “upregulated”). As used herein, expression of a miR gene product is “upregulated” when the amount of miR gene product in a sample from a subject is greater than the amount of the same gene product in a control (for example, a reference standard, a control cell sample, a control tissue sample).

In another embodiment, the level of the at least one miR gene product in the test sample is less than the level of the corresponding miR gene product in the control sample (i.e., expression of the miR gene product is “downregulated”). As used herein, expression of a miR gene is “downregulated” when the amount of miR gene product produced in a sample from a subject is less than the amount produced from the same gene in a control sample.

The relative miR gene expression in the control and normal samples can be determined with respect to one or more RNA expression standards. The standards can comprise, for example, a zero miR gene expression level, the miR gene expression level in a standard cell line, the miR gene expression level in unaffected samples of the subject, or the average level of miR gene expression previously obtained for a population of normal human controls (e.g., a control reference standard).

The level of the at least one miR gene product can be measured using a variety of techniques that are well known to those of skill in the art (e.g., quantitative or semi-quantitative RT-PCR, Northern blot analysis, solution hybridization detection). In a particular embodiment, the level of at least one miR gene product is measured by reverse transcribing RNA from a test sample obtained from the subject to provide a set of target oligodeoxynucleotides, hybridizing the target oligodeoxynucleotides to one or more miRNA-specific probe oligonucleotides (e.g., a microarray that comprises miRNA-specific probe oligonucleotides) to provide a hybridization profile for the test sample, and comparing the test sample hybridization profile to a hybridization profile generated from a control sample. An alteration in the signal of at least one miRNA in the test sample relative to the control sample is indicative of the subject either having, or being at risk for a particular disorder.

Also, a microarray can be prepared from gene-specific oligonucleotide probes generated from known miRNA sequences. The array may contain two different oligonucleotide probes for each miRNA, one containing the active, mature sequence and the other being specific for the precursor of the miRNA. The array may also contain controls, such as one or more mouse sequences differing from human orthologs by only a few bases, which can serve as controls for hybridization stringency conditions. tRNAs and other RNAs (e.g., rRNAs, mRNAs) from both species may also be printed on the microchip, providing an internal, relatively stable, positive control for specific hybridization. One or more appropriate controls for non-specific hybridization may also be included on the microchip. For this purpose, sequences are selected based upon the absence of any homology with any known miRNAs.

The microarray may be fabricated using techniques known in the art. For example, probe oligonucleotides of an appropriate length, e.g., 40 nucleotides, are 5′-amine modified at position C6 and printed using commercially available microarray systems, e.g., the GeneMachine OmniGrid™ 100 Microarrayer and Amersham CodeLink™ activated slides. Labeled cDNA oligomer corresponding to the target RNAs is prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions, e.g., 6× SSPE/30% formamide at 25° C. for 18 hours, followed by washing in 0.75× TNT at 37° C. for 40 minutes. At positions on the array where the immobilized probe DNA recognizes a complementary target cDNA in the sample, hybridization occurs. The labeled target cDNA marks the exact position on the array where binding occurs, allowing automatic detection and quantification. The output consists of a list of hybridization events, indicating the relative abundance of specific cDNA sequences, and therefore the relative abundance of the corresponding complementary miRs, in the patient sample. According to one embodiment, the labeled cDNA oligomer is a biotin-labeled cDNA, prepared from a biotin-labeled primer. The microarray is then processed by direct detection of the biotin-containing transcripts using, e.g., Streptavidin-Alexa647 conjugate, and scanned utilizing conventional scanning methods. Image intensities of each spot on the array are proportional to the abundance of the corresponding miR in the patient sample.

The use of the array has several advantages for miRNA expression detection. First, the global expression of several hundred genes can be identified in the same sample at one time point. Second, through careful design of the oligonucleotide probes, expression of both mature and precursor molecules can be identified. Third, in comparison with Northern blot analysis, the chip requires a small amount of RNA, and provides reproducible results using 2.5 μg of total RNA. The relatively limited number of miRNAs (a few hundred per species) allows the construction of a common microarray for several species, with distinct oligonucleotide probes for each. Such a tool allows for analysis of trans-species expression for each known miR under various conditions.

According to the expression profiling methods described herein, total RNA from a sample from a subject suspected of having a particular disorder can be quantitatively reverse transcribed to provide a set of labeled target oligodeoxynucleotides complementary to the RNA in the sample. The target oligodeoxynucleotides are then hybridized to a microarray comprising miRNA-specific probe oligonucleotides to provide a hybridization profile for the sample. The result is a hybridization profile for the sample representing the expression pattern of miRNA in the sample. The hybridization profile comprises the signal from the binding of the target oligodeoxynucleotides from the sample to the miRNA-specific probe oligonucleotides in the microarray. The profile may be recorded as the presence or absence of binding (signal vs. zero signal). More preferably, the profile recorded includes the intensity of the signal from each hybridization. The profile is compared to the hybridization profile generated from a normal control sample or reference sample. An alteration in the signal is indicative of the presence of, or propensity to develop, the particular disorder in the subject.

Other techniques for measuring miR gene expression are also within the skill in the art, and include various techniques for measuring rates of RNA transcription and degradation.

The invention also provides methods of diagnosing whether a subject has, or is at risk for developing, a particular disorder with an adverse prognosis. In this method, the level of at least one miR gene product, which is associated with an adverse prognosis in a particular disorder, is measured by reverse transcribing RNA from a test sample obtained from the subject to provide a set of target oligodeoxynucleotides. The target oligodeoxynucleotides are then hybridized to one or more miRNA-specific probe oligonucleotides (e.g., a microarray that comprises miRNA-specific probe oligonucleotides) to provide a hybridization profile for the test sample, and the test sample hybridization profile is compared to a hybridization profile generated from a control sample. An alteration in the signal of at least one miRNA in the test sample relative to the control sample is indicative of the subject either having, or being at risk for developing, a particular disorder with an adverse prognosis.

An “expression profile” or “hybridization profile” of a particular sample is essentially a fingerprint of the state of the sample; while two states may have any particular gene similarly expressed, the evaluation of a number of genes simultaneously allows the generation of a gene expression profile that is unique to the state of the cell. That is, normal samples may be distinguished from corresponding disorder-exhibiting samples. Within such disorder-exhibiting samples, different prognosis states (for example, good or poor long term survival prospects) may be determined. By comparing expression profiles of disorder-exhibiting samples in different states, information regarding which genes are important (including both upregulation and downregulation of genes) in each of these states is obtained.

The identification of sequences that are differentially expressed in disorder-exhibiting samples, as well as differential expression resulting in different prognostic outcomes, allows the use of this information in a number of ways. For example, a particular treatment regime may be evaluated (e.g., to determine whether a chemotherapeutic drug acts to improve the long-term prognosis in a particular subject). Similarly, diagnosis may be done or confirmed by comparing samples from a subject with known expression profiles. Furthermore, these gene expression profiles (or individual genes) allow screening of drug candidates that suppress the particular disorder expression profile or convert a poor prognosis profile to a better prognosis profile.

Alterations in the level of one or more miR gene products in cells can result in the deregulation of one or more intended targets for these miRs, which can lead to a particular disorder. Therefore, altering the level of the miR gene product (e.g., by decreasing the level of a miR that is upregulated in disorder-exhibiting cells, by increasing the level of a miR that is downregulated in disorder-exhibiting cells) may successfully treat the disorder.

Accordingly, the present invention encompasses methods of treating a disorder in a subject, wherein the expression of at least one miR gene product is regulated (e.g., downregulated, upregulated) in the cells of the subject. In one embodiment, the level of at least one miR gene product in a test sample is greater than the level of the corresponding miR gene product in a control or reference sample. In another embodiment, the level of at least one miR gene product in a test sample is less than the level of the corresponding miR gene product in a control sample. When the at least one isolated miR gene product is downregulated in the test sample, the method comprises administering an effective amount of the at least one isolated miR gene product, or an isolated variant or biologically-active fragment thereof, such that proliferation of the disorder-exhibiting cells in the subject is inhibited.

For example, when a miR gene product is downregulated in a cell in a subject, administering an effective amount of an isolated miR gene product to the subject can inhibit proliferation of the cell. The isolated miR gene product that is administered to the subject can be identical to an endogenous wild-type miR gene product that is downregulated in the cell or it can be a variant or biologically-active fragment thereof.

As defined herein, a “variant” of a miR gene product refers to a miRNA that has less than 100% identity to a corresponding wild-type miR gene product and possesses one or more biological activities of the corresponding wild-type miR gene product. Examples of such biological activities include, but are not limited to, inhibition of expression of a target RNA molecule (e.g., inhibiting translation of a target RNA molecule, modulating the stability of a target RNA molecule, inhibiting processing of a target RNA molecule) and inhibition of a cellular process associated with cancer and/or a myeloproliferative disorder (e.g., cell differentiation, cell growth, cell death). These variants include species variants and variants that are the consequence of one or more mutations (e.g., a substitution, a deletion, an insertion) in a miR gene. In certain embodiments, the variant is at least about 70%, 75%, 80%, 85%, 90%, 95%, 98%, or 99% identical to a corresponding wild-type miR gene product.

As defined herein, a “biologically-active fragment” of a miR gene product refers to an RNA fragment of a miR gene product that possesses one or more biological activities of a corresponding wild-type miR gene product. As described above, examples of such biological activities include, but are not limited to, inhibition of expression of a target RNA molecule and inhibition of a cellular process associated with such disorder. In certain embodiments, the biologically-active fragment is at least about 5, 7, 10, 12, 15, or 17 nucleotides in length. In a particular embodiment, an isolated miR gene product can be administered to a subject in combination with one or more additional treatments. Suitable treatments include, but are not limited to, chemotherapy, radiation therapy and combinations thereof (e.g., chemoradiation).

When the at least one isolated miR gene product is upregulated in the cells, the method comprises administering to the subject an effective amount of a compound that inhibits expression of the at least one miR gene product, such that proliferation of the disorder-exhibiting cells is inhibited. Such compounds are referred to herein as miR gene expression-inhibition compounds. Examples of suitable miR gene expression-inhibition compounds include, but are not limited to, those described herein (e.g., double-stranded RNA, antisense nucleic acids and enzymatic RNA molecules).

As described herein, when the at least one isolated miR gene product is upregulated in cells, the method comprises administering to the subject an effective amount of at least one compound for inhibiting expression of the at least one miR gene product, such that proliferation of such cells is inhibited.

The terms “treat”, “treating” and “treatment”, as used herein, refer to ameliorating symptoms associated with a disease or condition, including preventing or delaying the onset of the disease symptoms, and/or lessening the severity or frequency of symptoms of the disease, disorder or condition. The terms “subject”, “patient” and “individual” are defined herein to include humans, animals, such as mammals, including, but not limited to, primates, cows, sheep, goats, horses, dogs, cats, rabbits, guinea pigs, rats, mice or other bovine, ovine, equine, canine, feline, rodent, or murine species. In a preferred embodiment, the animal is a human.

As used herein, an “isolated” miR gene product is one that is synthesized, or altered or removed from the natural state through human intervention. For example, a synthetic miR gene product, or a miR gene product partially or completely separated from the coexisting materials of its natural state, is considered to be “isolated.” An isolated miR gene product can exist in a substantially-purified form, or can exist in a cell into which the miR gene product has been delivered. Thus, a miR gene product that is deliberately delivered to, or expressed in, a cell is considered an “isolated” miR gene product. A miR gene product produced inside a cell from a miR precursor molecule is also considered to be an “isolated” molecule. According to the invention, the isolated miR gene products described herein can be used for the manufacture of a medicament for treating a subject (e.g., a human).

Isolated miR gene products can be obtained using a number of standard techniques. For example, the miR gene products can be chemically synthesized or recombinantly produced using methods known in the art. In one embodiment, miR gene products are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., U.S.A.), Pierce Chemical (part of Perbio Science, Rockford, Ill., U.S.A.), Glen Research (Sterling, Va., U.S.A.), ChemGenes (Ashland, Mass., U.S.A.) and Cruachem (Glasgow, UK).

Alternatively, the miR gene products can be expressed from recombinant circular or linear DNA plasmids using any suitable promoter. Non-limiting examples of suitable promoters for expressing RNA from a plasmid include, e.g., the U6 or H1 RNA pol III promoter sequences, or the cytomegalovirus promoters. Selection of other suitable promoters is within the skill in the art. The recombinant plasmids of the invention can also comprise inducible or regulatable promoters for expression of the miR gene products in cells (e.g., cells exhibiting a particular disorder).

The miR gene products that are expressed from recombinant plasmids can be isolated from cultured cell expression systems by standard techniques. The miR gene products that are expressed from recombinant plasmids can also be delivered to, and expressed directly in, cells.

The miR gene products can be expressed from a separate recombinant plasmid, or they can be expressed from the same recombinant plasmid. In one embodiment, the miR gene products are expressed as RNA precursor molecules from a single plasmid, and the precursor molecules are processed into the functional miR gene product by a suitable processing system, including, but not limited to, processing systems extant within a cell.

Selection of plasmids suitable for expressing the miR gene products, methods for inserting nucleic acid sequences into the plasmid to express the gene products, and methods of delivering the recombinant plasmid to the cells of interest are within the skill in the art. For example, in certain embodiments, a plasmid expressing the miR gene products can comprise a sequence encoding a miR precursor RNA under the control of the CMV intermediate-early promoter. As used herein, “under the control” of a promoter means that the nucleic acid sequences encoding the miR gene product are located 3′ of the promoter, so that the promoter can initiate transcription of the miR gene product coding sequences.

The miR gene products can also be expressed from recombinant viral vectors. It is contemplated that the miR gene products can be expressed from two separate recombinant viral vectors, or from the same viral vector. The RNA expressed from the recombinant viral vectors can either be isolated from cultured cell expression systems by standard techniques, or can be expressed directly in cells (e.g., cells exhibiting a particular disorder).

In other embodiments of the treatment methods of the invention, an effective amount of at least one compound that inhibits miR expression can be administered to the subject. As used herein, “inhibiting miR expression” means that the production of the precursor and/or active, mature form of miR gene product after treatment is less than the amount produced prior to treatment. One skilled in the art can readily determine whether miR expression has been inhibited in cells using, for example, the techniques for determining miR transcript level discussed herein. Inhibition can occur at the level of gene expression (i.e., by inhibiting transcription of a miR gene encoding the miR gene product) or at the level of processing (e.g., by inhibiting processing of a miR precursor into a mature, active miR).

As used herein, an “effective amount” of a compound that inhibits miR expression is an amount sufficient to inhibit proliferation of cells in a subject suffering from a particular disorder. One skilled in the art can readily determine an effective amount of a miR expression-inhibiting compound to be administered to a given subject, by taking into account factors, such as the size and weight of the subject; the extent of disease penetration; the age, health and sex of the subject; the route of administration; and whether the administration is regional or systemic.

One skilled in the art can also readily determine an appropriate dosage regimen for administering a compound that inhibits miR expression to a given subject, as described herein. Suitable compounds for inhibiting miR gene expression include double-stranded RNA (such as short- or small-interfering RNA or “siRNA”), antisense nucleic acids, and enzymatic RNA molecules, such as ribozymes. Each of these compounds can be targeted to a given miR gene product and interfere with the expression (e.g., by inhibiting translation, by inducing cleavage and/or degradation) of the target miR gene product.

For example, expression of a given miR gene can be inhibited by inducing RNA interference of the miR gene with an isolated double-stranded RNA (“dsRNA”) molecule which has at least 90%, for example, at least 95%, at least 98%, at least 99%, or 100%, sequence homology with at least a portion of the miR gene product. In a particular embodiment, the dsRNA molecule is a “short or small interfering RNA” or “siRNA.”

In certain embodiments, administration of at least one miR gene product (and/or at least one compound for regulating miR expression) will affect the proliferation of cells (e.g., cells exhibiting a particular disorder) in a subject who has such disorder.

As used herein, to “alter the proliferation of cells exhibiting a particular disorder” can include one or more of: to kill the cells; to permanently or temporarily arrest or slow the growth of the cells; to reactive a desired gene expression in the cell; and, to modulate and/or reverse disease progression. For example, inhibition of cell proliferation can be inferred if the number of such cells in the subject remains constant or decreases after administration of the miR gene products or miR gene expression-regulating compounds. An inhibition of proliferation of cells exhibiting a particular disorder can also be inferred if the absolute number of such cells increases, but the rate of cell growth decreases.

A miR gene product or miR gene expression-regulating compound can also be administered to a subject by any suitable enteral or parenteral administration route. Suitable enteral administration routes for the present methods include, e.g., oral, rectal, or intranasal delivery. Suitable parenteral administration routes include, e.g., intravascular administration (e.g., intravenous bolus injection, intravenous infusion, intra-arterial bolus injection, intra-arterial infusion and catheter instillation into the vasculature); peri- and intra-tissue injection); subcutaneous injection or deposition, including subcutaneous infusion (such as by osmotic pumps); direct application to the tissue of interest, for example by a catheter or other placement device; and inhalation.

The miR gene products or miR gene expression-regulating compounds can be formulated as pharmaceutical compositions, sometimes called “medicaments,” prior to administering them to a subject, according to techniques known in the art. Accordingly, the invention encompasses pharmaceutical compositions for treating such disorder.

The present pharmaceutical compositions comprise at least one miR gene product or miR gene expression-regulating compound (or at least one nucleic acid comprising a sequence encoding the miR gene product or miR gene expression-regulating compound) (e.g., 0.1 to 90% by weight), or a physiologically-acceptable salt thereof, mixed with a pharmaceutically-acceptable carrier. In certain embodiments, the pharmaceutical composition of the invention additionally comprises one or more therapeutic agents. The pharmaceutical formulations of the invention can also comprise at least one miR gene product or miR gene expression-regulating compound (or at least one nucleic acid comprising a sequence encoding the miR gene product or miR gene expression-regulating compound), which are encapsulated by liposomes and a pharmaceutically-acceptable carrier.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include, e.g., physiologically biocompatible buffers (e.g., tromethamine hydrochloride), additions of chelants (such as, for example, DTPA or DTPA-bisamide) or calcium chelate complexes (such as, for example, calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid pharmaceutical compositions of the invention, conventional nontoxic solid pharmaceutically-acceptable carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can comprise any of the carriers and excipients listed above and 10-95%, preferably 25%-75%, of the at least one miR gene product or miR gene expression-inhibition compound (or at least one nucleic acid comprising sequences encoding them). A pharmaceutical composition for aerosol (inhalational) administration can comprise 0.01-20% by weight, preferably 1%-10% by weight, of the at least one miR gene product or miR gene expression-regulating compound (or at least one nucleic acid comprising a sequence encoding the miR gene product or miR gene expression-regulating compound) encapsulated in a liposome as described above, and a propellant. A carrier can also be included as desired; e.g., lecithin for intranasal delivery.

In one embodiment, the method comprises providing a test agent to a cell and measuring the level of at least one miR gene product associated with an altered expression levels in such cells. An alteration in the level of the miR gene product in the cell, relative to a suitable control (e.g., the level of the miR gene product in a control cell), is indicative of the test agent being therapeutic agent. Non-limiting examples of suitable agents include, but are not limited to, drugs (e.g., small molecules, peptides), and biological macromolecules (e.g., proteins, nucleic acids). The agent can be produced recombinantly, synthetically, or it may be isolated (i.e., purified) from a natural source. Various methods for providing such agents to a cell (e.g., transfection) are well known in the art, and several of such methods are described hereinabove. Methods for detecting the expression of at least one miR gene product (e.g., Northern blotting, in situ hybridization, RT-PCR, expression profiling) are also well known in the art.

The relevant teachings of all publications cited herein that have not explicitly been incorporated by reference, are incorporated herein by reference in their entirety.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

The miRs of interest are listed in public databases. In certain preferred embodiments, the public database can be a central repository provided by the Sanger Institute, microrna.sanger.ac.uk/sequences/ to which miR sequences are submitted for naming and nomenclature assignment, as well as placement of the sequences in a database for archiving and for online retrieval via the world wide web.

Generally, the data collected on the sequences of miRs by the Sanger Institute include species, source, corresponding genomic sequences and genomic location (chromosomal coordinates), as well as full length transcription products and sequences for the mature fully processed miRNA (miRNA with a 5′ terminal phosphate group). Another database can be the GenBank database accessed through the National Center for Biotechnology Information (NCBI) website, maintained by the National Institutes of Health and the National Library of Medicine. These databases are fully incorporated herein by reference.

| | miRBase Mature |
|---|---|
| miR | Sequence Accession # |
| hsa-let-7a* | MIMAT0004481 |
| hsa-let-7a-1 | MIMAT0000062 |
| hsa-let-7a-2 | MIMAT0000062 |
| hsa-let-7a-3 | MIMAT0000062 |
| hsa-let-7b | MIMAT0000063 |
| hsa-let-7b* | MIMAT0004482 |
| hsa-let-7c | MIMAT0000064 |
| hsa-let-7c* | MIMAT0004483 |
| hsa-let-7d | MIMAT0000065 |
| hsa-let-7d* | MIMAT0004484 |
| hsa-let-7e | MIMAT0000066 |
| hsa-let-7e* | MIMAT0004485 |
| hsa-let-7f-1 | MIMAT0000067 |
| hsa-let-7f-1* | MIMAT0004486 |
| hsa-let-7f-2 | MIMAT0000067 |
| hsa-let-7f-2* | MIMAT0004487 |
| hsa-let-7g | MIMAT0000414 |
| hsa-let-7g* | MIMAT0004584 |
| hsa-let-7i | MIMAT0000415 |
| hsa-let-7i* | MIMAT0004585 |
| hsa-mir-009-1 | MIMAT0000441 |
| hsa-mir-009-1* | MIMAT0000442 |
| hsa-mir-009-2 | MIMAT0000441 |
| hsa-mir-009-3 | MIMAT0000441 |
| hsa-mir-010a | MIMAT0000253 |
| hsa-mir-010a* | MIMAT0004555 |
| hsa-mir-015a | MIMAT0000068 |
| hsa-mir-015b | MIMAT0000417 |
| hsa-mir-015b* | MIMAT0004586 |
| hsa-mir-016-1 | MIMAT0000069 |
| hsa-mir-016-1* | MIMAT0004489 |
| hsa-mir-016-2 | MIMAT0000069 |
| hsa-mir-016-2* | MIMAT0004518 |
| hsa-mir-017-3-p | MIMAT0000071 |
| hsa-mir-017-5-p | MIMAT0000070 |
| hsa-mir-018a | MIMAT0000072 |
| hsa-mir-018a* | MIMAT0002891 |
| hsa-mir-019a | MIMAT0000073 |
| hsa-mir-019b-1 | MIMAT0000074 |
| hsa-mir-019b-1* | MIMAT0004491 |
| hsa-mir-019b-2 | MIMAT0000074 |
| hsa-mir-019b-2* | MIMAT0004492 |
| hsa-mir-020a | MIMAT0000075 |
| hsa-mir-020a* | MIMAT0004493 |
| hsa-mir-020b | MIMAT0001413 |
| hsa-mir-021 | MIMAT0000076 |
| hsa-mir-021* | MIMAT0004494 |
| hsa-mir-023a | MIMAT0000078 |
| hsa-mir-023a* | MIMAT0004496 |
| hsa-mir-023b | MIMAT0004587 |
| hsa-mir-024-1 | MIMAT0000080 |
| hsa-mir-024-1* | MIMAT0000079 |
| hsa-mir-024-2 | MIMAT0000080 |
| hsa-mir-024-2* | MIMAT0004497 |
| hsa-mir-025 | MIMAT0000081 |
| hsa-mir-025* | MIMAT0004498 |
| hsa-mir-026a-1 | MIMAT0000082 |
| hsa-mir-026a-1* | MIMAT0004499 |
| hsa-mir-026a-2 | MIMAT0000082 |
| hsa-mir-026a-2* | MIMAT0004681 |
| hsa-mir-026b | MIMAT0000083 |
| hsa-mir-026b* | MIMAT0004500 |
| hsa-mir-027a | MIMAT0000084 |
| hsa-mir-027a* | MIMAT0004501 |
| hsa-mir-027b | MIMAT0000419 |
| hsa-mir-027b* | MIMAT0004588 |
| hsa-mir-028-3p | MIMAT0004502 |
| hsa-mir-028-5p | MIMAT0000085 |
| hsa-mir-029a | MIMAT0000086 |
| hsa-mir-029a* | MIMAT0004503 |
| hsa-mir-029b-1 | MIMAT0000100 |
| hsa-mir-029b-1* | MIMAT0004514 |
| hsa-mir-029b-2 | MIMAT0000100 |
| hsa-mir-029b-2* | MIMAT0004515 |
| hsa-mir-029b-3 | MIMAT0000100 |
| hsa-mir-029c | MIMAT0000681 |
| hsa-mir-030a | MIMAT0000087 |
| hsa-mir-030a* | MIMAT0000088 |
| hsa-mir-030b | MIMAT0000420 |
| hsa-mir-030b* | MIMAT0004589 |
| hsa-mir-030c-1 | MIMAT0000244 |
| hsa-mir-030c-2 | MIMAT0000244 |
| hsa-mir-030c-2* | MIMAT0004550 |
| hsa-mir-030d | MIMAT0000245 |
| hsa-mir-030d* | MIMAT0004551 |
| hsa-mir-031 | MIMAT0000089 |
| hsa-mir-031* | MIMAT0004504 |
| hsa-mir-032 | MIMAT0000090 |
| hsa-mir-032* | MIMAT0004505 |
| hsa-mir-034a | MIMAT0000255 |
| hsa-mir-034a* | MIMAT0004557 |
| hsa-mir-092a-1 | MIMAT0000092 |
| hsa-mir-092a-1* | MIMAT0004507 |
| hsa-mir-093 | MIMAT0000093 |
| hsa-mir-093* | MIMAT0004509 |
| hsa-mir-095 | MIMAT0000094 |
| hsa-mir-096 | MIMAT0000095 |
| hsa-mir-096* | MIMAT0004510 |
| hsa-mir-098 | MIMAT0000096 |
| hsa-mir-099b | MIMAT0000689 |
| hsa-mir-099b* | MIMAT0004678 |
| hsa-mir-100 | MIMAT0000098 |
| hsa-mir-100* | MIMAT0004512 |
| hsa-mir-103-1 | MIMAT0000101 |
| hsa-mir-103-2 | MIMAT0000101 |
| hsa-mir-105-1 | MIMAT0000102 |
| hsa-mir-105-1* | MIMAT0004516 |
| hsa-mir-105-2 | MIMAT0000102 |
| hsa-mir-105-2* | MIMAT0004516 |
| hsa-mir-106a | MIMAT0000103 |
| hsa-mir-106a* | MIMAT0004517 |
| hsa-mir-106b | MIMAT0000680 |
| hsa-mir-106b* | MIMAT0004672 |
| hsa-mir-107 | MIMAT0000104 |
| hsa-mir-122 | MIMAT0000421 |
| hsa-mir-122* | MIMAT0004590 |
| hsa-mir-125a-3p | MIMAT0004602 |
| hsa-mir-125a-5p | MIMAT0000443 |
| hsa-mir-125b-1 | MIMAT0000423 |
| hsa-mir-125b-1* | MIMAT0004592 |
| hsa-mir-125b-2 | MIMAT0000423 |
| hsa-mir-125b-2* | MIMAT0004603 |
| hsa-mir-126 | MIMAT0000445 |
| hsa-mir-126* | MIMAT0000444 |
| hsa-mir-127-3p | MIMAT0000446 |
| hsa-mir-127-5p | MIMAT0004604 |
| hsa-mir-128-1 | MIMAT0000424 |
| hsa-mir-128-2 | MIMAT0000424 |
| hsa-mir-130a | MIMAT0000425 |
| hsa-mir-130a* | MIMAT0004593 |
| hsa-mir-130b | MIMAT0000691 |
| hsa-mir-130b* | MIMAT0004680 |
| hsa-mir-132 | MIMAT0000426 |
| hsa-mir-132* | MIMAT0004594 |
| hsa-mir-133a-1 | MIMAT0000427 |
| hsa-mir-133a-2 | MIMAT0000427 |
| hsa-mir-133b | MIMAT0000770 |
| hsa-mir-134 | MIMAT0000447 |
| hsa-mir-135b | MIMAT0000758 |
| hsa-mir-135b* | MIMAT0004698 |
| hsa-mir-140-3p | MIMAT0004597 |
| hsa-mir-140-5p | MIMAT0000431 |
| hsa-mir-142-3p | MIMAT0000434 |
| hsa-mir-142-5p | MIMAT0000433 |
| hsa-mir-143 | MIMAT0000435 |
| hsa-mir-143* | MIMAT0004599 |
| hsa-mir-145 | MIMAT0000437 |
| hsa-mir-145* | MIMAT0004601 |
| hsa-mir-146a | MIMAT0000449 |
| hsa-mir-146a* | MIMAT0004608 |
| hsa-mir-146b-3p | MIMAT0004766 |
| hsa-mir-146b-5p | MIMAT0002809 |
| hsa-mir-147 | MIMAT0000251 |
| hsa-mir-148a | MIMAT0000243 |
| hsa-mir-148a* | MIMAT0004549 |
| hsa-mir-148b | MIMAT0000759 |
| hsa-mir-148b* | MIMAT0004699 |
| hsa-mir-149 | MIMAT0000450 |
| hsa-mir-149* | MIMAT0004609 |
| hsa-mir-150 | MIMAT0000451 |
| hsa-mir-150* | MIMAT0004610 |
| hsa-mir-151-3p | MIMAT0000757 |
| hsa-mir-151-5p | MIMAT0004697 |
| hsa-mir-155 | MIMAT0000646 |
| hsa-mir-155* | MIMAT0004658 |
| hsa-mir-181a-1 | MIMAT0000256 |
| hsa-mir-181a-1* | MIMAT0000270 |
| hsa-mir-181a-2 | MIMAT0000256 |
| hsa-mir-181a-2* | MIMAT0004558 |
| hsa-mir-181b-1 | MIMAT0000257 |
| hsa-mir-181b-2 | MIMAT0000257 |
| hsa-mir-181d | MIMAT0002821 |
| hsa-mir-182 | MIMAT0000259 |
| hsa-mir-182* | MIMAT0000260 |
| hsa-mir-183 | MIMAT0000261 |
| hsa-mir-183* | MIMAT0004560 |
| hsa-mir-185 | MIMAT0000455 |
| hsa-mir-185* | MIMAT0004611 |
| hsa-mir-186 | MIMAT0000456 |
| hsa-mir-186* | MIMAT0004612 |
| hsa-mir-190 | MIMAT0000458 |
| hsa-mir-191 | MIMAT0000440 |
| hsa-mir-191* | MIMAT0001618 |
| hsa-mir-192 | MIMAT0000222 |
| hsa-mir-192* | MIMAT0004543 |
| hsa-mir-193a-3p | MIMAT0000459 |
| hsa-mir-193a-5p | MIMAT0004614 |
| hsa-mir-193b | MIMAT0002819 |
| hsa-mir-193b* | MIMAT0004767 |
| hsa-mir-195 | MIMAT0000461 |
| hsa-mir-195* | MIMAT0004615 |
| hsa-mir-196a* | MIMAT0004562 |
| hsa-mir-196a-1 | MIMAT0000226 |
| hsa-mir-196a-2 | MIMAT0000226 |
| hsa-mir-196b | MIMAT0001080 |
| hsa-mir-197 | MIMAT0000227 |
| hsa-mir-198 | MIMAT0000228 |
| hsa-mir-199a-3p | MIMAT0000232 |
| hsa-mir-199a-5p | MIMAT0000231 |
| hsa-mir-199a-5p | MIMAT0000231 |
| hsa-mir-199b-3p | MIMAT0004563 |
| hsa-mir-199b-5p | MIMAT0000263 |
| hsa-mir-200a | MIMAT0000682 |
| hsa-mir-200a* | MIMAT0001620 |
| hsa-mir-200b | MIMAT0000318 |
| hsa-mir-200b* | MIMAT0004571 |
| hsa-mir-200c | MIMAT0000617 |
| hsa-mir-200c* | MIMAT0004657 |
| hsa-mir-203 | MIMAT0000264 |
| hsa-mir-204 | MIMAT0000265 |
| hsa-mir-205 | MIMAT0000266 |
| hsa-mir-210 | MIMAT0000267 |
| hsa-mir-213 | MIMAT0000256 |
| hsa-mir-214 | MIMAT0000271 |
| hsa-mir-214* | MIMAT0004564 |
| hsa-mir-216a | MIMAT0000273 |
| hsa-mir-216b | MIMAT0004959 |
| hsa-mir-217 | MIMAT0000274 |
| hsa-mir-218-1 | MIMAT0000275 |
| hsa-mir-218-1* | MIMAT0004565 |
| hsa-mir-218-2 | MIMAT0000275 |
| hsa-mir-218-2* | MIMAT0004566 |
| hsa-mir-221 | MIMAT0000278 |
| hsa-mir-221* | MIMAT0004568 |
| hsa-mir-222 | MIMAT0000279 |
| hsa-mir-222* | MIMAT0004569 |
| hsa-mir-223 | MIMAT0000280 |
| hsa-mir-223* | MIMAT0004570 |
| hsa-mir-224 | MIMAT0000281 |
| hsa-mir-302a | MIMAT0000684 |
| hsa-mir-302a* | MIMAT0000683 |
| hsa-mir-302b | MIMAT0000715 |
| hsa-mir-302b* | MIMAT0000714 |
| hsa-mir-302c | MIMAT0000717 |
| hsa-mir-302c* | MIMAT0000716 |
| hsa-mir-302d | MIMAT0000718 |
| hsa-mir-302d* | MIMAT0004685 |
| hsa-mir-302e | MIMAT0005931 |
| hsa-mir-302f | MIMAT0005932 |
| hsa-mir-320a | MIMAT0000510 |
| hsa-mir-320b-1 | MIMAT0005792 |
| hsa-mir-320b-2 | MIMAT0005792 |
| hsa-mir-320c-1 | MIMAT0005793 |
| hsa-mir-320c-2 | MIMAT0005793 |
| hsa-mir-320d-1 | MIMAT0006764 |
| hsa-mir-320d-2 | MIMAT0006764 |
| hsa-mir-324-3p | MIMAT0000762 |
| hsa-mir-324-5p | MIMAT0000761 |
| hsa-mir-326 | MIMAT0000756 |
| hsa-mir-328 | MIMAT0000752 |
| hsa-mir-330-3p | MIMAT0000751 |
| hsa-mir-330-5p | MIMAT0004693 |
| hsa-mir-331-3p | MIMAT0000760 |
| hsa-mir-331-5p | MIMAT0004700 |
| hsa-mir-335 | MIMAT0000765 |
| hsa-mir-335* | MIMAT0004703 |
| hsa-mir-339-3p | MIMAT0004702 |
| hsa-mir-339-5p | MIMAT0000764 |
| hsa-mir-340 | MIMAT0004692 |
| hsa-mir-340* | MIMAT0000750 |
| hsa-mir-342-3p | MIMAT0000753 |
| hsa-mir-342-5p | MIMAT0004694 |
| hsa-mir-345 | MIMAT0000772 |
| hsa-mir-361-3p | MIMAT0004682 |
| hsa-mir-361-5p | MIMAT0000703 |
| hsa-mir-370 | MIMAT0000722 |
| hsa-mir-374a | MIMAT0000727 |
| hsa-mir-374b | MIMAT0004955 |
| hsa-mir-376a* | MIMAT0003386 |
| hsa-mir-376a-1 | MIMAT0000729 |
| hsa-mir-376a-2 | MIMAT0000729 |
| hsa-mir-376b | MIMAT0002172 |
| hsa-mir-376c | MIMAT0000720 |
| hsa-mir-378 | MIMAT0000732 |
| hsa-mir-378* | MIMAT0000731 |
| hsa-mir-382 | MIMAT0000737 |
| hsa-mir-411 | MIMAT0003329 |
| hsa-mir-411* | MIMAT0004813 |
| hsa-mir-423 | MIMAT0004748 |
| hsa-mir-423* | MIMAT0001340 |
| hsa-mir-425-3p | MIMAT0001343 |
| hsa-mir-425-5p | MIMAT0003393 |
| hsa-mir-432 | MIMAT0002814 |
| hsa-mir-432* | MIMAT0002815 |
| hsa-mir-433 | MIMAT0001627 |
| hsa-mir-484 | MIMAT0002174 |
| hsa-mir-485-3p | MIMAT0002176 |
| hsa-mir-485-5p | MIMAT0002175 |
| hsa-mir-486-3p | MIMAT0004762 |
| hsa-mir-486-5p | MIMAT0002177 |
| hsa-mir-487a | MIMAT0002178 |
| hsa-mir-487b | MIMAT0003180 |
| hsa-mir-532 | MIMAT0002888 |
| hsa-mir-532-5p | MIMAT0004780 |
| hsa-mir-539 | MIMAT0003163 |
| hsa-mir-574-3p | MIMAT0003239 |
| hsa-mir-574-5p | MIMAT0004795 |
| hsa-mir-584 | MIMAT0003249 |
| hsa-mir-628-3p | MIMAT0003297 |
| hsa-mir-628-5p | MIMAT0004809 |
| hsa-mir-643 | MIMAT0003313 |
| hsa-mir-660 | MIMAT0003338 |

*Biogenesis byproducts that are at low level, function unknown

1. A method of diagnosing or detecting susceptibility of a subject to one or more of a condition characterized by injury and tissue repair that transiently or permanently results in changes in one or more of an adaptive pathways and/or disease pathways,
wherein the disease pathway comprises lung fibrosis, including idiopathic pulmonary fibrosis (IPF) associated disease or an interstitial lung disease (ILD),
the method comprising:
determining the level of at least one miR gene product in the miR-17˜92 cluster in a sample from the subject, wherein the miR-17˜92 cluster comprises miR-19b, miR-17-3p, miR-17-5p, miR-18a, miR19a, miR-20a and miR-92; and
comparing the level of at least one miR gene product in the sample to a control,
wherein an alteration in the level of the at least one miR gene product in the sample from the subject, relative to that of the control, is diagnostic or prognostic of such disease.

2. (canceled)

3. (canceled)

4. (canceled)

5. (canceled)

6. (canceled)

7. The method of claim 1, wherein the miR comprises miR-19b.

8. The method of claim 7, wherein at least one other miR in the miR-17˜92 cluster is used in combination with miR-19b.

9. (canceled)

10. The method of claim 1, wherein the control is selected one or more of:
a reference standard;
the level of the at least one miR gene product from a subject that does not have the disease; and
the level of the at least one miR gene product from a sample of the subject that does not exhibit such disease.

11. The method of claim 1, wherein the subject is a human.

12. (canceled)

13. (canceled)

14. A method of inhibiting progression or proliferation of idiopathic pulmonary fibrosis associated disorder in a subject, comprising:
introducing into at least one cell of the subject one or more agents which alter expression and/or activity of at least one miR in the miR-17˜92 cluster within the cell, wherein the miR-17˜92 cluster comprises miR-19b, miR-17-3p, miR-17-5p, miR-18a, miR19a, miR-20a and miR-92, and
maintaining the cells under conditions in which the one or more agents: inhibits expression or activity of the miR; enhances expression or activity of one or more target genes of the miR; or, results in a combination thereof, thereby inhibiting progression or proliferation of the disease or disorder.

15. The method of claim 14, wherein the miR comprises miR-19b.

16. The method of claim 15, wherein at least one other miR in the miR-17˜92 cluster is used in combination with miR-19b.

17. The method of claim 14, wherein the cell is a human cell.

18. A method of identifying a therapeutic for to, idiopathic pulmonary fibrosis (IPF) agent, comprising:
providing a test agent to a cell and measuring the level of at least one miR in the miR-17˜92 cluster associated with an altered expression levels in the cells, wherein the miR-17˜92 cluster comprises miR-19b, miR-17-3p, miR-17-5p, miR-18a, miR19a, miR-20a and miR-92,
wherein an alteration in the level of the miR in the cell, relative to a suitable control cell, is indicative of the test agent being a therapeutic agent.

19. The method of claim 19, wherein the miR comprises miR-19b.

20. The method of claim 19, wherein at least one other miR in the miR-17˜92 cluster is used in combination with miR-19b.

21. A method for regulating levels of one or more proteins in a subject having, or at risk of developing idiopathic pulmonary fibrosis (IPF) associated disorder, wherein the at least one protein comprises one or more of: c-myc, CTGF, TSP1, HDAC4, the method comprising:
altering the expression of at least one miR gene product in the miR-17˜92 cluster lung cells in the subject, wherein the miR-17˜92 cluster comprises miR-19b, miR-17-3p, miR-17-5p, miR-18a, miR19a, miR-20a and miR-92.

22. The method of claim 21, wherein the miR comprises miR-19b.

23. The method of claim 22, wherein at least one other miR in the miR-17˜92 cluster is used in combination with miR-19b.

24. (canceled)

25. (canceled)

26. The method of claim 21, wherein the subject has idiopathic pulmonary fibrosis (IPF).

27. The method of claim 21, wherein the subject has an interstitial lung disease (ILD).

28. A method for assessing prognosis in a subject with idiopathic pulmonary fibrosis associated disorder, comprising:
determining an level of at least one miR in the miR-17˜92 cluster which alters expression of one or more of the protein levels of for c-myc, CTGF and HDAC4 as a prognostic indicator of disease progression, wherein the miR-17˜92 cluster comprises miR-19b, miR-17-3p, miR-17-5p, miR-18a, miR19a, miR-20a and miR-92.

29. The method of claim 28, wherein the miR comprises miR-19b.

30. The method of claim 29, wherein at least one other miR in the miR-17˜92 cluster is used in combination with miR-19b.

31. (canceled)

32. A method for assessing prognosis in a subject with idiopathic pulmonary fibrosis- or ILD-associated disorder, comprising:
determining an altered expression of one or more of the protein levels as a prognostic indicator of disease progression,
wherein the at least one protein comprises one or more of: c-myc, CTGF, TSP1, HDAC4, wherein at least miR-19b and mir20a are down regulated with increasing severity of disease in patients with IPF.

33. The method of claim 32, wherein the miR comprises miR-19b.

34. The method of claim 33, wherein at least one other miR in the miR-17˜92 cluster is used in combination with miR-19b.

35. A method for altering the expression of a target gene in a subject having, or at risk or developing idiopathic pulmonary fibrosis (IPF), comprising: inducing expression of one or more miRs in the miR-17˜92 clusters in cells in the subject, wherein the miR-17˜92 cluster comprises miR-19b, miR-17-3p, miR-17-5p, miR-18a, miR19a, miR-20a and miR-92.

36. The method of claim 35, wherein the miR comprises miR-19b.

37. The method of claim 36, wherein at least one other miR in the miR-17˜92 cluster is used in combination with miR-19b.

38. The method of claim 37, comprising inducing expression by transient transfection in IPF fibroblast cells in the subject sufficient to alter expression of at least one target and/or to change at least one gene networks, to expression those present in normal fibroblast cells, wherein one or more miRs of the miR-17˜92 cluster downregulate expression of one or more genes selected from: CTGF, TGFβ, MMPs, VEGF, Col1a, and thrombospondin-1 (TSP1).

39. (canceled)

40. (canceled)

41. A method for treating idiopathic pulmonary fibrosis (IPF) fibroblasts in lung cells in a subject, comprising introducing one or more miRs in the miR-17˜92 cluster into the cells in an amount sufficient to recover a proliferative and younger phenotype in the cells, wherein the miR-17˜92 cluster comprises miR-19b, miR-17-3p, miR-17-5p, miR-18a, miR19a, miR-20a and miR-92.

42. The method of claim 41, wherein the miR comprises miR-19b.

43. The method of claim 42, wherein at least one other miR in the miR-17˜92 cluster is used in combination with miR-19b.

44. A method for enhancing wound healing in lung cells a subject having or at risk of developing a fibrotic disease of an organ, including but not limited to, idiopathic pulmonary fibrosis (IPF) or facilitating wound repair in an adaptive setting, comprising transfecting cells with one or more miRs in the miR-17˜92 cluster, wherein the miR-17˜92 cluster comprises miR-19b, miR-17-3p, miR-17-5p, miR-18a, miR19a, miR-20a and miR-92.

45. The method of claim 44, wherein the miR comprises miR-19b.

46. The method of claim 45, wherein at least one other miR in the miR-17˜92 cluster is used in combination with miR-19b.

47. The method of claim 44, wherein the cells comprise lung cells.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. A method for detecting changes in myofibroblast production and/or detecting alterations in epithelial cell-to-mesenchymal cell transition in a subject having, or at risk of developing idiopathic pulmonary fibrosis (IF), comprising measuring levels of one or more miRs in the miR17˜92 cluster in lung cells in the subject, wherein the miR-17˜92 cluster comprises miR-19b, miR-17-3p, miR-17-5p, miR-18a, miR19a, miR-20a and miR-92.

69. The method of claim 68, wherein at least one of miR-19b and miR-20a are down regulated with increasing severity of disease in patients with a fibrotic disease of an organ, including but not limited to IPF.

70. The method of claim 68, wherein the miR comprises miR-19b.

71. The method of claim 70, wherein at least one other miR in the miR-17˜92 cluster is used in combination with miR-19b.
